(19) United States
(12) Patent Application Publication
(10) Pub. No.: US 2020/0000940 A1
(43) Pub. Date: Jan. 2, 2020

(54) LAYERED SENSORS AND METHODS OF USING

(71) Applicant: Profusa, Inc., South San Francisco, CA (US)

(72) Inventors: Kate BALACONIS, College Station, TX (US); Scott NICHOLS, College Station, TX (US); Sierra GUIDRY, Bryan, TX (US); Yu ZHANG, College Station, TX (US); Ryan SCHWELLER, College Station, TX (US)

(21) Appl. No.: 16/459,077

(22) Filed: Jul. 1, 2019

Related U.S. Application Data
(63) Continuation of application No. PCT/US19/39932, filed on Jun. 28, 2019.
(60) Provisional application No. 62/692,161, filed on Jun. 29, 2018.
(60) Provisional application No. 62/692,161, filed on Jun. 29, 2018.

(51) Int. Cl. A61K 49/00

(52) U.S. Cl. CPC A61K 49/0021; A61K 49/0015

(57) ABSTRACT

Layered implantable sensors are described herein. Layered sensors described herein may include one or more analyte sensing populations. The one or more analyte sensing populations may detect different analytes, or different concentrations of the same analyte, for example. The layered sensors may include a reference population. The reference population may, or may not, be analyte sensing. As described herein, the first sensing population may be separated from a second sensing population (and/or a reference population) by a passive layer.

### CROSS REFERENCE TO RELATED APPLICATIONS

This application is a non-provisional of, and claims priority to, U.S. Patent Application No. 62/692,161, filed Jun. 29, 2018, the entire disclosure of which is hereby incorporated by reference. This application is a continuation of international patent application no. PCT/US19/39932, filed Jun. 28, 2019, which claims priority to U.S. Patent Application No. 62/692,161, the entire disclosure of each of which is hereby incorporated by reference.

### FIELD

The present disclosure is in the field of luminescent dyes, polymers and sensors.

### BACKGROUND

This application is related to U.S. patent application Ser. No. 16/023,906, filed Jun. 29, 2019, which claims priority to U.S. Patent Application No. 62/526,961, each of which is entitled “Multi-Analyte Sensing Tissue-Integrating Sensors,” the entire disclosure of each of which is hereby incorporated by reference in its entirety.

Currently, sensors exist that can be implanted in tissue. For example, sensors exist that can be implanted a few millimeters under the skin. In such sensors, luminescent dyes are typically used to measure the concentration of an analyte of interest. These sensors may use one or more additional sensing elements to provide an internal reference and/or may include multiple sensing elements for multi-analyte sensing. In some cases, the internal reference or other sensing elements may be influenced by other sensing components. Accordingly, a need layered sensors that eliminate or minimize cross-sensitivity and crosstalk between sensing elements.

### BRIEF DESCRIPTION OF THE DRAWINGS

FIG. 1 is a schematic illustration of an example of a sensing mechanism of lactate sensors described herein.

FIG. 2 shows performance of an embodiment of a layered lactate and oxygen sensor (n=4) to (A) an oxygen modulation and (B) a lactate modulation. FIG. 2 illustrates that the oxygen sensor portion (square) responds during the oxygen modulation but remains stable during the lactate modulation, during which oxygen is maintained at a fixed concentration. FIG. 2 further illustrates that the lactate sensor portion (circle) responds to both the lactate and oxygen modulations because it contains an oxygen-sensitive dye. Mean and standard deviations are shown.

FIG. 3 shows the change in phosphorescent lifetime measurements from the oxygen sensing layer of an embodiment of a sensor between 0 and 24 mM lactate. FIG. 3 illustrates that as the number of layers increases, the response of the oxygen sensor decreases close to zero, indicating a small amount of cross-sensitivity impacting the oxygen sensing layer.

FIGS. 4A, 4B, and 4C show schematics of exemplary sensors including a coating, a first sensing population, and a second sensing population, as described herein.

### DETAILED DESCRIPTION

Layered implantable sensors are described herein. Layered sensors described herein may include one or more analyte sensing populations. The one or more analyte sensing populations may detect different analytes, or different concentrations of the same analyte, for example. The layered sensors may include a reference population. The reference population may, or may not, be analyte sensing.

As described herein, the first sensing population may be separated from a second sensing population (and/or a reference population) by a passive layer. The passive layer may include polymers. The passive layer may be a coating or tubing.

The passive layer separating the different sensing layers of a multi-layer sensor provides several advantages, including minimizing or eliminating cross-talk between the signals from the different sensing layers.

In some embodiments described herein, a sensor may include more than one layer. In an embodiment, a central layer may include a sensing population. In an aspect, the central layer may include more than one sensing populations. In a further aspect, the central layer may include more than one sensing populations, wherein at least one sensing population is a reference population.

In some embodiments, the central layer can include a polymer and/or one or more sensing populations. The central layer may be formed from a precursor solution. In some embodiments, the precursor solution for the central layer may include up to 100% monomer and/or polymer by weight. In an aspect, the precursor solution for the central layer may include greater than 99% monomer and/or polymer by weight, and less than 1% sensing population, cosolvents, and/or crosslinking components by weight. In some embodiments, the precursor solution for the central layer may greater than 90% monomer and/or polymer by weight, and less than 10% sensing population, cosolvents, and/or crosslinking components by weight. In some embodiments, the precursor solution for the central layer may include greater than 80% monomer and/or polymer by weight, and less than 20% sensing population, cosolvents, and/or crosslinking components by weight. In some embodiments, the precursor solution for the central layer may include greater than 70% monomer and/or polymer by weight, and less than 30% sensing population, cosolvents, and/or crosslinking components by weight. In some embodiments, the precursor solution for the central layer may include, greater than 60% monomer and/or polymer by weight, and less than 40% sensing population, cosolvents, and/or crosslinking components by weight. In some embodiments, the precursor solution for the central layer may include greater than 50% monomer and/or polymer by weight, and less than 50% sensing population, cosolvents, and/or crosslinking components by weight. In some embodiments, the precursor solution for the central layer may include greater than 40% monomer and/or polymer by weight, and less than 60% sensing population, cosolvents, and/or crosslinking components by weight. In some embodiments, the precursor solution for the central layer may include greater than 30% monomer and/or polymer by weight, and less than 70% sensing population, cosolvents, and/or crosslinking components by weight. In some embodiments, the precursor solution for the central layer may include greater than 20% monomer and/or polymer by weight, and less than 80% sensing population, cosolvents, and/or crosslinking components by weight. In some embodiments, the precursor solution for the central layer may include greater than 10% monomer and/or polymer by weight, and less than 90% sensing population, cosolvents, and/or crosslinking components by weight.

In some embodiments, the central layer may be completely or partially encapsulated by a second layer. The second layer may be formed from a precursor solution. In some embodiments, the second layer may be a passive layer. Similarly stated, in embodiments in which the second layer is a passive layer, the second layer may include a polymer and/or other inactive components, and may not include a sensing population and/or a reference population, e.g., the precursor solution for the second layer may include up to 100% monomer and/or polymer by weight. In other embodiments, the second layer can be an active layer. Thus, in some embodiments, the precursor solution for the second layer may include greater than 99% monomer and/or polymer by weight, and less than 1% sensing population, cosolvents, and crosslinking components by weight. In some embodiments, the precursor solution for the second layer may include greater than 90% monomer and/or polymer by weight, and less than 10% sensing population, cosolvents, and crosslinking components by weight. In some embodiments, the precursor solution for the second layer may include greater than 80% monomer and/or polymer by weight, and less than 20% sensing population, cosolvents, and crosslinking components by weight. In some embodiments, the precursor solution for the second layer may include greater than 70% monomer and/or polymer by weight, and less than 30% sensing population, cosolvents, and crosslinking components by weight. In some embodiments, the precursor solution for the second layer may include greater than 60% monomer and/or polymer by weight, and less than 40% sensing population, cosolvents, and crosslinking components by weight. In some embodiments, the precursor solution for the second layer may include greater than 50% monomer and/or polymer by weight, and less than 50% sensing population, cosolvents, and crosslinking components by weight. In some embodiments, the precursor solution for the second layer may include greater than 40% monomer and/or polymer by weight, and less than 60% sensing population, cosolvents, and crosslinking components by weight. In some embodiments, the precursor solution for the second layer may include greater than 30% monomer and/or polymer by weight, and less than 70% sensing population, cosolvents, and crosslinking components by weight In some embodiments, the precursor solution for the second layer may include greater than 20% monomer and/or polymer by weight, and less than 80% sensing population, cosolvents, and crosslinking components by weight. In some embodiments, the precursor solution for the second layer may include greater than 10% monomer and/or polymer by weight, and less than 90% sensing population, cosolvents, and crosslinking components by weight. Third and/or subsequent layers can have similar compositions.

In an embodiment, the second layer may be a sensing layer. In an aspect, the second layer may include a sensing population.

The sensor can include any suitable number of layers. For example, a second layer of the sensor, which partially and/or completely encapsulates a central layer, can be partially and/or completely encapsulated by a third layer. The third layer can be partially and/or completely encapsulated by a fourth layer, and so forth.

In some embodiments sensing layers can be separated by one or more passive layers. Similarly stated, in some such embodiments each layer containing a sensing and/or reference population can be separated from other layers containing a sensing and/or reference population by one or more layers that are devoid of a sensing and/or reference population.

### Sensing Layer

One or more layers of the sensor may be a sensing layer. Sensing layers may provide for continuous or semi-continuous collection of data of various biochemical analytes. The sensing layer may detect an analyte, such as a biochemical analyte, and produce a detectable signal that is associated with and/or correlated to a concentration of the analyte. The signal may be an optical signal.

Non-limiting examples of analytes that may be detected by the sensing layer include oxygen, reactive oxygen species, glucose, lactate, pyruvate, cortisol, creatinine, urea, sodium, magnesium, calcium, potassium, vasopressin, hormones (e.g., Luteinizing hormone), pH, cytokines, chemokines, eicosanoids, insulin, leptins, small molecule drugs, ethanol, myoglobin, nucleic acids (RNAs, DNAs), fragments, polypeptides, single amino acids and the like.

The sensing layer may, for example, utilize reversible binding ligands and/or chemistries for analyte detection. The sensing layer may, for example, utilize irreversible or consumptive chemistries for analyte detection. The sensing layer may include one or more sensing moieties, for example, to detect one or more analytes of interest. Suitable sensing moieties include, but are not limited to: analyte binding molecules (e.g. glucose binding proteins), competitive binding molecules (e.g. phenylboronic acid based chemistries), analyte specific enzymes (e.g. lactate oxidase, glucose oxidase, dehydrogenase), ion sensitive materials (e.g. ionophores), or other analyte sensitive molecules (e.g. oxygen sensitive dyes such as porphyrins). In an embodiment, the layered sensors described herein may be used to detect an analyte that may be detected with an oxidase. In an aspect, the sensing moiety may include an oxidase. Exemplary oxidases include but are not limited to naturally occurring oxidases, genetically engineered oxidases, monooxygenases, glucose oxidase, lactate oxidase, pyruvate oxidase, ethanol oxidase, bilirubin oxidase, and histamine oxidase. Exemplary dehydrogenases include but are not limited to glucose dehydrogenase and lactate dehydrogenase. In an embodiment, the sensing moiety may be a combination of an oxidase and dehydrogenase, including the combination of lactate oxidases and lactate dehydrogenases. In an embodiment, the sensing moiety may be an analyte binding protein. Exemplary analyte binding proteins include but are not limited to concanavalin A, glucose binding protein, and lactate binding protein. In an embodiment, the sensing moiety may be a chemical binding structure. In an embodiment, the recognition element may be an antibody. In an embodiment, the sensing moiety may be a non-enzymatic catalyst. In an embodiment, the sensing moiety may be an aptamer.

In an embodiment, a sensing layer may include more than one sensing moieties. In an aspect, the more than one sensing moieties may be collocated in the sensing layer. In an aspect, the more than one sensing moieties may be located at different portions of the sensing layer. In an aspect, the different sensing moieties may be separated spatially or through the use of particles, microparticles or nanoparticles.

In an embodiment, the sensing moieties may be commercially available or may be produced by a user. Protein or enzyme-based sensing moieties may be naturally occurring, may be recombinant, may contain mutations, or may have post transcriptional modifications such as glycosylation, or the like. In an embodiment, the sensing moiety may be a monomer, dimer, trimer, tetramer, or octamer.

In an embodiment, the sensing moiety may be physically entrapped or chemically bound within the sensor layer. In an embodiment, the sensing moiety may be attached to a polymer, such through a covalent or non-covalent linkage. In an embodiment, the sensing moiety may not be chemically conjugated to the polymer. In another embodiment, the sensing moiety may be attached to the surface of the sensor, such as via covalent or non-covalent linkages. In yet another embodiment, the sensing moiety may be present within the sensor through more than one of the above means, e.g., sensing moiety may be attached to the polymer via a covalent linkage and physically entrapped within the sensor. In an embodiment, the sensing moiety may be on the surface of the sensor and also within the sensor. In an embodiment, the sensor may be covered by an exterior coating. In an embodiment, the sensing moiety may be encapsulated into particles, microparticles or nanoparticles. In an embodiment, the sensing moiety may be in solution, with or without a polymer.

In an embodiment, the sensing layer may include an optically detectable dye. An optical property of the dye may be altered when the sensing moiety detects an analyte. For example, an intensity of the optical signal and/or an emitted wavelength of the dye may be altered in the presence of an analyte.

In an embodiment, the optically detectable dye may be covalently, or non-covalently, bound to a polymer. In an embodiment, the optically detectable dye may be physically entrapped within a polymer. In an aspect, the polymer-bound-optically detectable dye may be optically distinguishable from the optically detectable dye that isn't bound to the polymer, or is bound to a different polymer. For example, the polymer-bound-optically detectable dye may have a longer decay than that of the optically detectable dye that isn't bound to the polymer.

In an embodiment, the optically detectable dye may be an oxygen sensitive dye. In an embodiment, the oxygen sensitive dye may be a porphyrin dye. The oxygen sensitive dye may be a NIR porphyrin molecule. In an embodiment, the oxygen sensitive dye may be selected from one described in U.S. Pat. No. 9,375,494, which is hereby incorporated by reference herein.

In an embodiment, the sensing moiety may be an oxidase and the optically detectable dye may be an oxygen sensitive dye. The oxidase and the oxygen sensitive dye may be collocated in the sensing layer.

In an embodiment, the sensing moiety and dye may be located in different layers. In an embodiment, the layers may be adjacent. In an embodiment, the sensing moiety is an oxidase and the optically detectable dye may be an oxygen sensitive dye.

In an embodiment, the oxygen sensitive dye may be covalently attached to the polymer. In an embodiment, the oxygen sensitive dye may be covalently attached to the oxidase. In an embodiment, the oxygen sensitive dye may be non-covalently bound to the polymer.

In an embodiment, the sensing layer may include one or more monomers or polymers that form a scaffold. In an aspect, the polymer may be a hydrogel. The polymers of the scaffold may be the same as the polymers bound to the sensing moiety, or the polymers of the scaffold may be different from the polymers bound to the sensing moiety. The polymers of the scaffold may be the same as the polymers bound to the optically detectable dye, or the polymers of the scaffold may be different from the polymers bound to the optically detectable dye.

In an embodiment, the sensing moiety may be labeled with a reporter (e.g., one or more fluorophores, one or more gold particles, one or more quantum dots and/or one or more single-walled carbon nanotubes). Sensing moieties may also create a signal through swelling, optical diffraction, change in absorbance FRET, and/or quenching.

The sensing layer may include other molecules besides sensing molecules, such as carrier molecules/polymers (e.g. the sensing layer may include polyethylene glycol nanospheres, alginate particles or other carrier materials that contain sensing molecules). The sensing layer may also contain reference molecules or stabilizing molecules that do not sense any analytes, but that serves as calibrators (e.g., a reference dye or any substance that provides a reference signal to which the signal modulated by the analyte of interest may be compared for calibration) or stabilizer (e.g. catalase, any free-radical scavenger which helps preserve the sensing moieties or other stabilizer). The sensing layer may contain drugs that slowly elute from the layer (e.g. dexamethasone, insulin).

The sensing layer may include thermally responsive material, pressure-responsive material, biodegradable material or materials that swell, shrink, change optical properties, or change other measurable properties in response to a stimulus.

In an embodiment, the sensing layer may include other scaffold materials, as described herein. In an embodiment, the sensing layer may include other scaffold materials and may not include a polymer. In an embodiment, the sensing layer may include other scaffold materials and may also include one or more polymers.

In an embodiment, sensors designed to measure different concentrations of an analyte are contemplated. For example, separate sensing layers can contain distinct sensing populations operable to measure different concentration ranges of a single analyte or different analytes. For example, a first sensing layer can be configured to produce a signal that is associated with and/or correlated to a concentration of the analyte when the analyte concentration is low (e.g., below a first threshold), while a second sensing layer can be configured to produce a signal that is associated with and/or correlated to a concentration of the analyte when the analyte concentration is high (e.g., above a second threshold). For example, the first sensing layer may become saturated or otherwise produce a signal that is uncorrelated to analyte concentration when the analyte concentration reaches too high (e.g., above a third threshold that is greater than the first threshold). The second sensing layer can have a minimum detection threshold. Similarly stated, the second sensing layer can be configured to produce a signal that is associated with and/or correlated to a concentration of the analyte when the analyte concentration is above the minimum detection threshold, but may not be operable to produce a signal that is accurately correlated to analyte concentration when the concentration is below the minimum detection threshold. The minimum detection threshold of the second sensing layer can be greater than the first threshold and/or less than the third threshold.

In an embodiment, the sensing moiety may be a lactate sensing protein. In an embodiment, the lactate sensing protein may be lactate oxidase, and the detected analyte may be lactate.

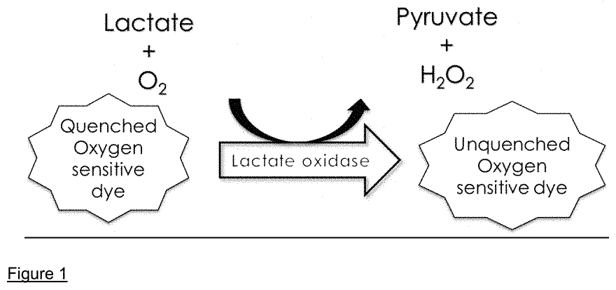

In an embodiment, lactate sensors described herein may include one or more polymers, one or more lactate oxidases, and one or more oxygen sensitive dyes. Additionally, the lactate sensors may further include one or more oxygen sensitive reference dye. Without being bound by a particular mechanism, it is believed that in the lactate sensors described herein, as the lactate is enzymatically converted, oxygen is consumed by the enzyme (FIG. 1). The sensors measure the amount of oxygen, and the depletion of oxygen is directly related to the lactate concentration for a given oxygen concentration.

Exemplary lactate oxidases include, but are not limited to, lactate oxidase and its homologues, including lactate 2-monooxygenase, lactate oxidative decarboxylase, lactic oxygenase, lactate oxygenase, lactic oxidase, L-lactate oxidase, L-lactate monooxygenase, L-lactate 2-monooxygenase, and lactate monooxygenase. Lactate oxidases may be derived from different species including Aerococcus viridans, Pediococcus species, Mycobacterium species including Mycobacterium smegmatis and Mycobacterium phlei, Streptococcus species including Streptococcus pyogenes and Streptococcus iniae, Enterococcus species, and Zymomonas mobilis.

An embodiment relates to a sensor including two or more lactate sensing populations separated by a passive layer. One lactate sensing population is configured to measure lactate at a first percentage of oxygen, and a second lactate sensing population is configured to measure lactate at a second percentage of oxygen. The first sensing population may be separated from the second sensing population by a passive layer. The sensor can further include additional lactate sensing populations that are configured to measure lactate at different percentages of oxygen. Each lactate sensing population includes one or more polymers, one or more lactate oxidases, and one or more oxygen sensitive dyes. As shown in FIG. 1, lactate oxidases consume oxygen and convert lactate to either pyruvate and hydrogen peroxide or acetate, carbon dioxide, and water. The reduction of oxygen in the vicinity of the enzyme can be measured by using an oxygen-sensitive dye, such as a porphyrin dye. These dye molecules are quenched in the presence of oxygen, so the reduction of oxygen by the action of lactate oxidases causes an increase in luminescence and phosphorescent lifetime. Luminescence and phosphorescent lifetimes from the oxygen-sensitive dyes is thus proportional to the concentration of lactate in the sensor.

An embodiment relates to a sensor including a lactate sensing layer, a passive layer, and a reference layer. One exemplary configuration may be: lactate sensing layer-passive layer-reference layer. A second exemplary configuration may be: reference layer-passive layer-lactate sensing layer. In an aspect, the reference layer may be configured to detect oxygen, allowing for the determination of the local concentration of oxygen.

An embodiment relates to a sensor including a first analyte sensing layer that is configured to detect a first concentration of an analyte, a second analyte sensing layer that is configured to detect a second concentration of an analyte, a first passive layer, a second passive layer, and a reference layer. One exemplary configuration may be: first analyte sensing layer-first passive layer-second analyte sensing layer-second passive layer-reference layer. A second exemplary configuration may be: first analyte sensing layer-first passive layer-reference layer-second passive layer-second analyte sensing layer. A third exemplary configuration may be: reference layer-first passive layer-first analyte sensing layer-second passive layer-second analyte sensing layer. In an aspect, the reference layer may be configured to detect oxygen, allowing for the determination of the local concentration of oxygen. In an aspect, the sensing layers may detect lactate.

In an embodiment, the optical emission spectrum of the first sensing layer may be distinguished from the optical emission spectrum of the second sensing layer. In an embodiment, the optical emission spectrum of a reference layer may be distinguished from the optical emission spectrum of the one or more sensing layers. Similarly stated, each sensing layer and/or reference layer can, in some embodiments, be configured to emit an optical signal having a different characteristic wavelength and/or time-response behavior.

In an embodiment, the sensing moiety of a first sensing layer may be attached (either covalently or non-covalently) to a first polymer, and the sensing moiety of a second sensing layer may be attached (either covalently or non-covalently) to a second polymer.

An embodiment relates to a sensor including a lactate sensing layer and a layer that detects a different analyte.

As described herein, the measurement of analytes by the described sensors may not require implanted electronics.

### Passive Layer

In an embodiment, a first sensing layer may be fully or partially encapsulated by a passive layer. The passive layer may include a coating and/or tubing. References to coatings and/or tubing described herein should be understood as referring to a passive layer. In an aspect, the passive layer may completely or partially enclose the first sensing layer and first sensing population.

The passive layer may include the same or different polymer materials as those in the sensing layers. In an aspect, the passive layer may separate first sensing population and second sensing population by between 0 and 5 mm. In an embodiment, the passive layer may be between 0.1 um and 2 mm thick and/or wide. In an embodiment, the passive layer may be greater than 0.1 um thick and/or wide. In an embodiment, the passive layer may be greater than 10 um thick and/or wide. In an embodiment, the total sensor length may be between 1 and 5 mm. In another embodiment the ratio of the lengths and/or thicknesses of the sensing layer to the total sensor length may be between 0.4 and 1.0.

In an embodiment, the passive layer may include one or more monomers or polymers selected from the group consisting of (Table 3): PU-SG80A (Lubrizol Inc.), PU D3 (AdvanSource Biomaterials Inc.), PU D640 (AdvanSource Biomaterials Inc.), polymethylmethacrylate (PMMA), polycaprolactone (PCL), PU OP770 (Lubrizol Inc.), PU SG-85A (Lubrizol Inc.), PU EG-93A (Lubrizol Inc.), and polycarbonate (PC). In an embodiment, the passive layer may include one or more monomers or polymers selected from the group consisting of: polycarbonate, PU-SG80A, and PU EG-93A. In an aspect, the passive layer may be PU EG-93A.

In an embodiment, the passive layer may include one or more compounds selected from the group identified in (Table 3), polyethylene (PE), polyurethane (PU), silicone, and polymethylpentene (TPX). In an embodiment, the tubing may include one or more compounds selected from the group consisting of: polymethylpentene or polyethylene. In an aspect, the passive layer may include polymethylpentene.

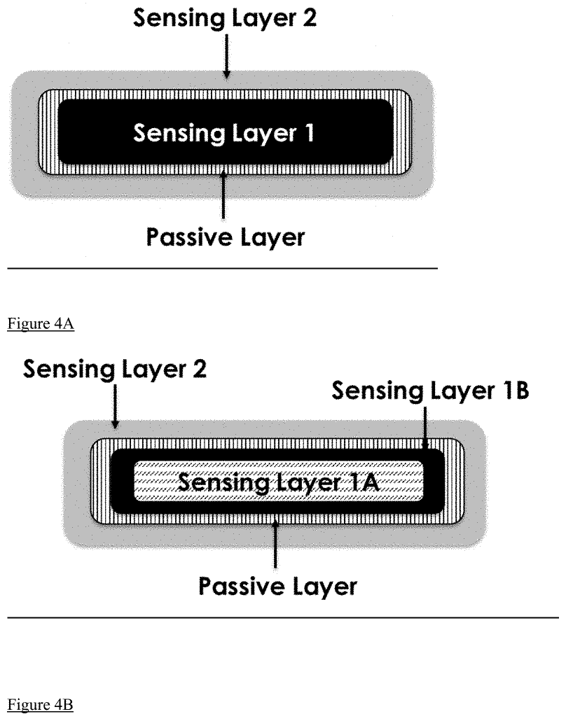

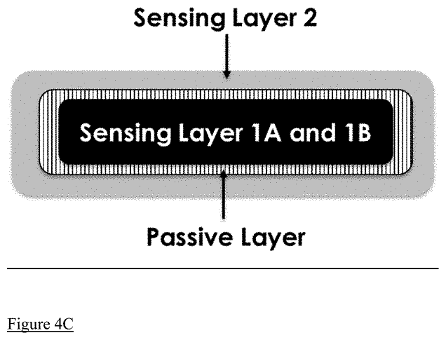

In an embodiment, a first sensing population may be separated from a second sensing population by a passive layer, as shown, for example, in FIG. 4A. The passive layer may include tubing, coating, or a combination of tubing and coating. In an embodiment, the tubing and/or coating may only partially encapsulate the sensing population. In an embodiment, the tubing and/or coating may not cover the ends of the sensing population. In an embodiment, a tubing and/or coating may encapsulate the sensing layer; the sensing layer may include a polymer scaffold and both a sensing population and reference population.

In an aspect, the tubing may be pre-formed, and the central (e.g., sensing and/or reference) layer may be formed inside the tubing. In an aspect, the tubing may be pre-formed, and the central layer may be placed inside the tubing. In an aspect, the tubing may be partially pre-formed, and the central layer may be placed inside the tubing. In an aspect, the ends of the tubing may remain open.

In an embodiment, sensors may include multiple sensing and/or reference portions separated by multiple layers of coatings and/or tubing. In an embodiment, the coatings and/or tubing may not be the same for each layer (e.g., different passive layers may be constructed of different materials). In some embodiments, the sensor includes at least two sensing layers, in which at least one sensing layer fully or partially encompasses at least one passive layer. In some embodiments, a sensing layer that encompasses and/or is encompassed by a passive layer can be a reference layer, such as a reference layer configured to detect oxygen.

In some embodiment, the passive layer may include other scaffold materials, as described herein. For example, the passive layer may include other scaffold materials and may not include a polymer. As an alternative example, the passive layer may include other scaffold materials and may also include one or more polymers.

### Reference Population

In an embodiment, a second sensing layer may include a reference population. In certain embodiments, the reference layer may include additional moieties (e.g., non-sensing or additional sensing moieties different from the sensing moieties), for example reference (or calibration) moieties. Reference moieties (which also may be referred to as calibration moieties) include, but are not limited to, dyes, fluorescent particles, lanthanides, nanoparticles, microspheres, quantum dots or other additives or elements of the implant whose signal does not change due to the presences of the analyte (e.g., glucose) that the sensing layers are configured to detect. Chaudhary et al. (2009) Biotechnology and Bioengineering 104(6):1075-1085, which is hereby incorporated herein by reference in its entirety, describes some suitable reference moieties. Fluctuations in the reference (calibration) signal(s) can be used to correct or calibrate the sensing signal(s). For example, in an embodiment in which sensing moieties are configured to detect lactate or other suitable analyte by detecting a local change in oxygen concentration caused by a reaction between the analyte and an enzyme and/or catalyst (e.g., an oxidase such as lactate oxidase), the reference layer may also include an additional oxygen-sensitive dye that serves as a reference for the amount of locally present oxygen.

In an embodiment, the oxygen reference dye may be a porphyrin dye. The oxygen reference dye may be a NIR porphyrin ring molecule. In an embodiment, the oxygen reference dye may include the same type of chemistry as the oxygen-sensitive dye. The oxygen reference dye may be selected from one described in U.S. Pat. No. 9,375,494, which is hereby incorporated by reference herein.

The oxygen reference dye may be covalently or non-covalently attached to a polymer. The polymer and the one or more oxygen reference dyes may form an oxygen reference population. The polymer of the oxygen reference population may be the same or different from the polymer of the scaffold. In an embodiment, one or more of the oxygen reference dye populations may be microspheres, nanospheres, microparticles, nanoparticles, and the like.

In an embodiment, the sensing moiety of a sensing layer may be attached to a first polymer, and the sensing moiety of a reference layer may be attached to a different polymer. In an embodiment, the sensing moiety of the sensing layer and the sensing moiety of the additional sensing layer may both emit optical signals that have similar, or the same wavelengths; however, with the addition of the first polymer to the sensing moiety of the sensing layer and the addition of a different polymer to the sensing moiety of the reference layer, the two sensing moieties emit optical signals that are distinguishable (e.g., have different wavelengths).

In an embodiment, a first sensing layer and a second sensing layer may be configured to detect different concentrations of the same analyte. In an aspect, the first sensing layer may configured to detect the analyte when the analyte is present in at least a first concentration, and the second sensing layer may be configured to detect the analyte when the analyte is present in at least a second concentration that is higher than the first concentration. In some such embodiments, the first sensing layer may be saturated and/or otherwise insensitive to the analyte when present at concentrations greater than a third concentration, the third concentration can be greater than, less than, or equal to the second concentration.

In an embodiment, a second sensing layer may include other scaffold materials, as described herein. In an embodiment, a second sensing layer may include other scaffold materials and may not include a polymer. In an embodiment, the second sensing layer may include other scaffold materials and may also include one or more polymers.

In some embodiments, the first sensing population may be configured to detect a first analyte and the second sensing population may be configured to detect a second analyte. In some such embodiments, the second sensing population may serve as a reference for the first population.

### Polymers

In an aspect, the one or more polymers (e.g., included within a sensing layer, a reference layer, and/or a passive layer) may be formed from one or more methacrylate or acrylate monomers, one or more methacrylate or acrylate comonomers, and one or more methacrylate or acrylate crosslinkers.

In an embodiment, the one or more monomers and/or polymers of the sensing layer(s), reference layer(s) and/or passive layer(s) may include the group consisting of (Tables 1 and 2): 2-hydroxyethylmethacrylate (HEMA), butylmethacrylate (BMAcrylate), hydroxypropyl methacrylate (HPMA), methyl methacrylate (MMA), n-hexylacrylate (nHA), [2-(methacryloyloxy)ethyl]dimethyl-(3-sulfopropyl)ammonium hydroxide, [2-(methacryloyloxy)ethyl]dimethyl-(3-sulfopropyl)ammonium hydroxide/acrylamide (1:1), 1,1,1,3,3,3-hexafluoroisopropyl acrylate, 2-(tert-butylamino)ethyl methacrylate, 2,2,2-trifluoroethyl methacrylate, 2,2,3,3,4,4,4-heptafluorobutyl methacrylate, 2,2,3,3,4,4,5,5-octafluoro-1,6-hexyldimethacrylate, 2,2,3,3-tetrafluoropropyl methacrylate, 2,2,3,4,4,4-hexafluorobutyl methacrylate, 2-carboxyethyl acrylate, 2-fluoroethyl methacrylate, 2-methacryloyloxyethyl phosphorylcholine, 3-chloro-2-hydroxypropyl methacrylate, benzyl methacrylate, ethylene glycol dicyclopentenyl ether methacrylate, lauryl methacrylate, o-nitrobenzyl methacrylate, pentafluorobenzyl methacrylate, Polyurethane D640 (AdvanSource Biomaterials Inc), dimethylacrylamide (DMA), N-(2-hydroxyethyl) methacrylamide, N-Isopropylacrylamide, poly(ethylene glycol) diacrylamide, acrylamide). In an embodiment, the monomer and the comonomer are not the same. In an aspect, the monomers and/or polymers may be selected from the group consisting of: HEMA, nHA, HPMA, 2,2,3,3,4,4,4-heptafluorobutyl methacrylate, 2-carboxyethyl acrylate, [2-(methacryloyloxy)ethyl]dimethyl-(3-sulfopropyl)ammonium hydroxide, [2-(acryloyloxy)ethyl]trimethylammonium chloride, 2-hydroxyethyl methacrylate, 2,2,2-trifluoroethyl methacrylate, methyl methacrylate, ethylene glycol dicyclopentenyl ether methacrylate, benzyl methacrylate, 2-fluoroethyl methacrylate, pentafluorobenzyl methacrylate, and Polyurethane D640. In an aspect, the monomers and/or polymers may be selected from the group consisting of: HPMA, nHA, HPMA, 2-carboxyethyl acrylate, [2-(methacryloyloxy)ethyl]dimethyl-(3-sulfopropyl)ammonium hydroxide, 2-fluoroethyl methacrylate, pentafluorobenzyl methacrylate, [2-(acryloyloxy)ethyl]trimethylammonium chloride, and Polyurethane D640.

In an embodiment, the one or more monomers and/or polymers of the sensing layer(s), reference layer(s), and/or passive layer(s) may include monomers and/or polymers selected from the group consisting of: N,N′-methylenebis(acrylamide), bisphenol A glycerolate diacrylate (BPADA), ethylene glycol dimethacrylate (EGDMA), 1,6-hexanediol diacrylate (HDDA), neopentyl glycol diacrylate (NPDA), pentaerythritol triacrylate (PEA3), pentaerythritol tetraacrylate (PEA4), poly(etheylene glycol) diacrylate (PEGDA), diurethane dimethacrylate (UDMA), and tetraethylene glycol dimethacrylate (TEGDMA). In an embodiment, the crosslinker may be selected from the group consisting of: bisphenol A glycerolate diacrylate (BPADA), ethylene glycol dimethacrylate (EGDMA), 1,6-hexanediol diacrylate (HDDA), neopentyl glycol diacrylate (NPDA), pentaerythritol triacrylate (PEA3), pentaerythritol tetraacrylate (PEA4), poly(etheylene glycol) diacrylate (PEGDA), and diurethane dimethacrylate (UDMA), trimethylolpropane triacrylate, tetraethylene glycol dimethacrylate, poly(ethylene glycol) diacrylate (Mn=700), and N,N′-methylenebis(acrylamide). In an aspect, monomers and/or polymers may be EGDMA, tetraethylene glycol dimethacrylate, poly(ethylene glycol) diacrylate (Mn=700 and N,N′-methylenebis(acrylamide).

The monomers and/or polymers of embodiments described herein may be described by the weight and/or volume percentage of three primary monomers and/or polymers in the precursor solution. Prior to polymerization, these monomers may comprise 10-90% volume of the precursor solution. In one embodiment, these monomers may comprise 30-80% volume of the precursor solution. In one embodiment, these monomers may comprise 50-70% volume of the precursor solution. In one embodiment, these monomers may comprise 70% volume of the precursor solution. The remaining volumetric components may be sensing elements, dyes, co-solvents, crosslinkers that incorporate into the polymer.

In particular embodiments, the weight percentage of component 1 as compared to the other primary monomers and/or polymers (Table 1) may be: 40 to 100% w/w. In an embodiment, weight percentage of component 1 (Table 1) may be: 60 to 80% w/w. In an embodiment, weight percentage of component 1 (Table 1) may be: 60 to 75% w/w.

In particular embodiments, the weight percentage of component 2 as compared to the other primary monomers and/or polymers (Table 1) may be: 0 to 50% w/w. In an embodiment, weight percentage of component 2 (Table 1) may be: 10 to 30% w/w. In an embodiment, weight percentage of component 1 (Table 1) may be: 15 to 30% w/w.

In particular embodiments, the weight percentage of component 3 as compared to the other primary monomers and/or polymers (Table 1) may be: 0 to 25% w/w. In an embodiment, weight percentage of component 2 (Table 1) may be: 5 to 15% w/w. In an embodiment, weight percentage of component 1 (Table 1) may be: 8 to 11% w/w.

In particular embodiments, the weight percentage of component 1 as compared to the other primary monomers and/or polymers (Table 2) may be: 40 to 100% w/w. In an embodiment, weight percentage of component 1 (Table 2) may be: 50 to 98% w/w. In an embodiment, weight percentage of component 1 (Table 2) may be: 55 to 96% w/w.

In particular embodiments, the weight percentage of component 2 as compared to the other primary monomers and/or polymers (Table 2) may be: 0 to 50% w/w. In an embodiment, weight percentage of component 2 (Table 2) may be: 1.5 to 45% w/w. In an embodiment, weight percentage of component 1 (Table 2) may be: 3.5 to 40% w/w.

In particular embodiments, the weight percentage of component 3 as compared to the other primary monomers and/or polymers (Table 2) may be: 0 to 25% w/w. In an embodiment, weight percentage of component 2 (Table 2) may be: 0.1 to 10% w/w. In an embodiment, weight percentage of component 1 (Table 2) may be: 0.1 to 2.5% w/w.

In some embodiments, one or more monomers and/or polymers may be formed from one or more acrylamide or methacrylamide monomers, one or more acrylamide or methacrylamide comonomers, and one or more acrylamide or methacrylamide crosslinkers. In an embodiment, the acrylamide or methacrylamide monomers and comonomers may be selected from the group consisting of: dimethacrylamide, butylmethacrylamide, 2-hydroxypropylmethacrylamide, and N-(2-hydroxyethyl)methacrylamide. In an embodiment, the crosslinker may be selected from the group consisting of: methylenebisacrylamide, ethylenebisacrylamide, and polyethylene glycol diacrylamide.

### Other Scaffold Materials

In some embodiments, the sensing layers, the passive layer(s), and/or the reference layer(s) may include one or more other scaffold materials. The other scaffold materials may be materials that are not polymers. Exemplary other scaffold materials include, but are not limited to: mesoporous and macroporous materials from carbon, silica, alumina, metal oxides, and ceramics. Exemplary other scaffold materials include, but are not limited to: mesoporous carbon, activated carbon, mesoporous silica or alumina, mesoporous metal oxides, and mesoporous ceramics. inorganic hydrogels (e.g. nanoclay hydrogel), inorganic/organic hybrid hydrogels (e.g. nanocomposite hydrogels).

### Sensor Design

In some embodiments, a second sensing population can completely or partially enclose a passive layer that in turn completely or partially encloses a first sensing population. An example of this design is shown in FIG. 4A.

In an embodiment, the dye for the first sensing population may be the same or similar to the dye from the second sensing (and/or reference) population. In an embodiment, the first sensing population may include a first polymer and the second sensing population may include a second polymer. In an embodiment, the emission spectrum of the dye of the first sensing population may be distinguished from the emission spectrum of the dye from the second sensing population. In an embodiment, a signal associated with the first sensing population may be distinguished from a signal associated with the second sensing population based on temporal characteristics. For example, a decay rate of the luminescence (e.g., the phosphorescence) of the dye of the first sensing population may differ from a decay rate of the luminescence (e.g., the phosphorescence) of the dye of the second sensing population.

In an embodiment, the first sensing population may include an oxidase and the second sensing population may include an oxygen sensing portion. The oxygen sensing portion can serve as a reference to determine the local concentration of oxygen, and this information may be used as to calibrate the oxidase sensor. This calibration may occur as part of an algorithm functioning in a reader, or other device external to the user.

In an embodiment, the first sensing population may detect lactate. The lactate sensing population may include both lactate sensing protein and oxygen-sensitive dye, which can function together to detect lactate according to the reaction in FIG. 1. In an aspect, the lactate sensing protein and oxygen-sensitive dye may be collocated, as shown in FIG. 4A. In an aspect, the lactate sensing protein and oxygen-sensitive dye may be near each other or beside each other. In an aspect, the lactate sensing protein may surround the oxygen-sensitive dye, as shown in FIG. 4B. In an aspect, the oxygen-sensitive dye may surround the lactate sensing protein, as shown in FIG. 4C.

In an embodiment, a lactate sensor may be separated from the oxygen reference by a coating or tubing (e.g., a passive layer).

Embodiments in which a central layer is a sensing layer, the middle layer is a passive layer, and an outer layer is an additional sensing layer are described above. Additional embodiments are contemplated. For example, a central layer may be the additional sensing layer, a middle layer may be the passive layer, and an outer layer may be the sensing layer.

Embodiments in which the sensor includes three layers are described above. Embodiments including additional layers (e.g., fourth, fifth, etc.) are also contemplated. For example, an additional (e.g., second) passive layer may encapsulate an additional (e.g., second) sensing layer, and a third sensing layer and/or reference layer may encapsulate the additional passive layer. The layers may be stacked in any configuration that allows for one or more passive layers to separate the first sensing layers, the additional sensing layers, and the reference layers. For example, one contemplated configuration is: first sensing layer-first passive layer-second sensing layer-second passive layer-reference layer. An additional contemplated configuration is: first sensing layer-first passive layer-reference layer-second passive layer-second sensing layer. An additional contemplated configuration is: reference layer-first passive layer-first sensing layer-second passive layer-second sensing layer.

In an embodiment, the different sensing layers detect different concentrations of the same analyte. For example, a sensor may have a first sensing layer that is configured to detect a first concentration of an analyte; a first passive layer that encapsulates the first sensing layer; a second sensing layer that is configured to detect a second concentration of the analyte and encapsulates the passive layer; a second passive layer that encapsulates the second sensing layer; and a reference layer that encapsulates the second passive layer.

In an embodiment, a single sensing layer may include more than one sensing moiety. For example, a single sensing layer may be configured to detect more than one analyte. As another example, a single sensing layer may be configured to detect more than one concentration of the same analyte.

In an embodiment, the sensor may be 1-10 mm in length. The sensor may be 0.25-2 mm in diameter, width or height. In an embodiment, the sensor may be rod-shaped, spherical, block-like, cube-like, disk-shaped, cylindrical, oval, round, random or non-random configurations of fibers and the like. In an embodiment, the sensor may be a microsphere or a nanosphere.

In an embodiment, one sensor may include two or more sensing populations. These two or more sensing populations may be in distinct portions of the sensor. In an aspect, each of the two or more sensing populations may detect different analytes. In an aspect, each of the two or more sensing populations may detect different concentrations of the same analyte. In an aspect, a first sensing population of a sensor may measure lactate at a first concentration of oxygen, and a second sensing population of the sensor may measure lactate at a second concentration of oxygen. In an embodiment, the second concentration of oxygen may be higher than the first concentration of oxygen. In an embodiment, at least one of the concentrations of oxygen may be a physiological concentration of oxygen.

In an embodiment, one or more of the sensing populations may include microspheres, nanospheres, microparticles, nanoparticles, and the like. In an embodiment, the scaffold of the sensor may include a polymer that be different from, or the same as, the polymer in a sensing population.

In an embodiment, the sensor may include distinct layers where the sensing recognition element is physically entrapped or chemically bound to or within specific layers of the sensor. In a further embodiment, the sensor may include additional layers; the additional layers may provide other features such as mechanical strength, elasticity, conductivity or other properties. The additional layers may detect different analytes, different concentrations of the same analyte. The additional layers may include a reference dye.

In an embodiment, multiple sensors containing the same or different sensing populations may be implanted near each other. For example, one or more sensors containing (optionally, exclusively) a first sensing population may be implanted near one or more sensors containing (optionally, exclusively) a second sensing population. For example, one or more sensors containing only the oxygen reference population may be implanted near the one or more sensors containing only a first sensing population and/or a second sensing population configured to detect one or more other (e.g., non-oxygen) analytes (e.g., lactate, different concentrations of lactate, etc.). In an aspect, one sensor may include multiple sensing populations. For example, one or more sensors containing the first sensing population and the second sensing population may be implanted near one or more sensors containing a third sensing population. For example, one or more sensors containing both a first sensing population and a second sensing population (e.g., sensing populations configured to detect different concentrations of an analyte or different analytes) may be implanted near one or more sensors containing one or more oxygen reference populations. In an aspect, a sensor may include one or more sensing populations and one or more reference populations. The sensors may be implanted in a particular design, such as a ring, or another geometry.

### Methods of Making Layered Sensors

Methods of making layered sensors are described herein. In an embodiment, a first layer is provided, a passive layer is applied over the first layer, and then an outer layer is applied over the passive layer.

In an embodiment, the first layer may be a sensing layer and the outer layer may be a reference layer.

In an embodiment, the passive layer may be applied over the first layer. In an aspect, the passive layer may be polymerized prior to the application to the first layer. In an aspect, the passive layer may be polymerized after application to the first layer.

In an embodiment, the outer layer may be applied over the passive layer. In an aspect, the outer layer may be polymerized prior to the application to the passive layer. In an aspect, the outer layer may be polymerized after application to the first layer.

In an embodiment, layered sensors described herein may be fabricated using polymerization techniques, including free radical-based, living radical, or living chain polymerization reactions as well as stepwise or step growth polymerizations such as reversible addition-fragmentation chain transfer (RAFT) or atom-transfer radical-polymerization (ATRP). In an embodiment, step growth polymerization can be achieved through the use of Cu(I) catalyzed azide-alkyne cycloaddition (CuAAC), strain-promoted azide-alkyne cycloaddition, thiol-ene photocoupling, Diels-Alder reaction, inverse electron demand Diels-Alder reaction, tetrazole-alkene photo-click reaction, oxime reaction, Michael-type addition including thiol-Michael addition and amine-Michael addition, and aldehyde-hydrazide coupling, chelation.

In an embodiment, polymers described herein may be fabricated using other techniques that include ionic crosslinking, hydrophobic-hydrophobic interactions, hydrogen bonding, polar-polar interactions, and chelation. Other exemplary methods include incorporating sensing populations into mesoporous or microporous materials or into semi-permeable membranes.

In an embodiment, incorporation of a passive layer may be achieved by injecting or loading the sensing population into tubing and initiating scaffold formation within the tubing. In addition, sensing populations and scaffolds may be created outside of the tubing then manually loaded into the tubing. This process of loading sensing populations into the tubing may involve chemical or thermal swelling and subsequent deswelling of the tubing. In an embodiment, the tubing may be loaded with a combination of additional scaffold material and a preformed sensing population then polymerized in place. In a separate embodiment, the tubing may be loaded with a scaffold material and a preformed sensor which can be sealed via melting of the tubing, chemical bonding of the tubing, or the addition of coatings.

In an embodiment, passive layers may be added by dip coating sensing populations one or multiple times into the passive layer material. In an embodiment, passive layers may be added by spin coating. In an embodiment, passive layers are preformed in a mold. In an embodiment, passive layers are added through in-situ crosslinking. In an embodiment, passive layers may be formed through the use of Cu(I) catalyzed azide-alkyne cycloaddition (CuAAC), strain-promoted azide-alkyne cycloaddition, thiol-ene photocoupling, Diels-Alder reaction, inverse electron demand Diels-Alder reaction, tetrazole-alkene photo-click reaction, oxime reaction, Michael-type addition including thiol-Michael addition and amine-Michael addition, and aldehyde-hydrazide coupling. In an embodiment, passive layers are directly attached to sensing populations through polymerization.

### Properties

In an embodiment, the scaffold of the sensor may be constructed such that it has conduits, pores or pockets that are hollow or filled with degradable, angiogenic, or other substances (e.g. stem cells). In some embodiments, the sensor, once in the body, can be configured such that biodegradation of the material filling the conduits, pores or pockets, may create space for tissue, including capillaries, to integrate with the material. The degradable material that initially fills the conduits, pores or pockets may enhance vessel growth or tissue growth within the scaffold. This architecture may promote new vessel formation and maintains healthy viable tissue within and around the implant.

### Methods of Using Layered Sensors

Layered sensors as described herein are useful in the monitoring of a number of conditions. The layered sensors may be placed subcutaneously, surrounding tissue of muscle, subcutaneous fat, dermis, in muscle, in skin, in the limbs, sternum, neck, ear, brain, or other locations.

The layered sensors described herein may be useful in monitoring trauma, sepsis, exercise physiology/performance optimization, overall health monitoring, skin grafts, wound healing, shock, and other disease states as described in Andersen et al. (2013) Mayo Clin Proc 88 (10): 1127-1140, which is hereby incorporated herein by reference in its entirety.

### Measurements of Sensors Described Herein

After initial sensor injection, measurements can be collected non-invasively through luminescent NIR signals with a specially designed optical reader. In an embodiment, the optical reader is located outside of the body. These continuous analyte sensors have the potential to transform the field of analyte monitoring by providing non-invasive, real-time, continuous analyte measurements in a user-friendly, cost-effective format.

### EXAMPLES

TABLE 1
Lactate sensor compositions (w/w % of monomer and/or
polymer content of major components)

| | | | wt % | wt % | wt % |
|---|---|---|---|---|---|
| Component 1 | Component 2 | Component 3 | cmpt 1 | cmpt 2 | cmpt 3 |
| poly(ethylene glycol) | | | 100.00 | | |
| diacrylamide (Mn = 3700) | | | | | |
| 2-hydroxyethyl | ethylene glycol | | 90.18 | 9.82 | |
| methacrylate | dimethacrylate | | | | |
| 2-hydroxyethyl | tetraethylene glycol | | 97.96 | 2.04 | |
| methacrylate | dimethacrylate | | | | |
| hydroxypropyl | ethylene glycol | | 90.04 | 9.96 | |
| methacrylate | dimethacrylate | | | | |
| N-(2-hydroxyethyl) | tetraethylene glycol | | 77.12 | 22.88 | |
| methacrylamide | dimethacrylate | | | | |
| N-(2-hydroxyethyl) | tetraethylene glycol | | 88.35 | 11.65 | |
| methacrylamide | dimethacrylate | | | | |
| pentafluorobenzyl | ethylene glycol | | 92.27 | 7.73 | |
| methacrylate | dimethacrylate | | | | |
| 2-hydroxyethyl | hydroxypropyl | ethylene glycol | 45.43 | 44.68 | 9.89 |
| methacrylate | methacrylate | dimethacrylate | | | |
| 2-hydroxyethyl | hydroxypropyl | ethylene glycol | 63.42 | 26.73 | 9.86 |
| methacrylate | methacrylate | dimethacrylate | | | |
| 2-hydroxyethyl | hydroxypropyl | ethylene glycol | 63.42 | 26.73 | 9.86 |
| methacrylate | methacrylate | dimethacrylate | | | |
| 2-hydroxyethyl | hydroxypropyl | ethylene glycol | 64.05 | 26.99 | 8.96 |
| methacrylate | methacrylate | dimethacrylate | | | |
| 2-hydroxyethyl | hydroxypropyl | ethylene glycol | 72.37 | 17.79 | 9.84 |
| methacrylate | methacrylate | dimethacrylate | | | |
| 2-hydroxyethyl | ethylene glycol | hydroxypropyl methacrylate | 76.85 | 14.76 | 8.40 |
| methacrylate | dimethacrylate | | | | |
| 2-hydroxyethyl | ethylene glycol | hydroxypropyl methacrylate | 81.29 | 9.83 | 8.88 |
| methacrylate | dimethacrylate | | | | |
| 2-hydroxyethyl | methyl methacrylate | ethylene glycol | 47.64 | 41.99 | 10.37 |
| methacrylate | | dimethacrylate | | | |
| 2-hydroxyethyl | methyl methacrylate | ethylene glycol | 65.23 | 24.63 | 10.14 |
| methacrylate | | dimethacrylate | | | |
| 2-hydroxyethyl | methyl methacrylate | ethylene glycol | 73.73 | 16.24 | 10.03 |
| methacrylate | | dimethacrylate | | | |
| 2-hydroxyethyl | ethylene glycol | methyl methacrylate | 82.05 | 9.92 | 8.03 |
| methacrylate | dimethacrylate | | | | |
| 2-hydroxyethyl | n-hexyl acrylate | ethylene glycol | 66.22 | 23.49 | 10.30 |
| methacrylate | | dimethacrylate | | | |
| 2-hydroxyethyl | n-hexyl acrylate | ethylene glycol | 70.01 | 24.83 | 5.16 |
| methacrylate | | dimethacrylate | | | |
| 2-hydroxyethyl | n-hexyl acrylate | ethylene glycol | 74.46 | 15.41 | 10.13 |
| methacrylate | | dimethacrylate | | | |
| 2-hydroxyethyl | ethylene glycol | n-hexyl acrylate | 77.88 | 14.96 | 7.16 |
| methacrylate | dimethacrylate | | | | |
| 2-hydroxyethyl | n-hexyl acrylate | ethylene glycol | 78.49 | 11.46 | 10.05 |
| methacrylate | | dimethacrylate | | | |
| 2-hydroxyethyl | n-hexyl acrylate | ethylene glycol | 78.66 | 16.27 | 5.07 |
| methacrylate | | dimethacrylate | | | |
| 2-hydroxyethyl | poly(ethylene glycol) | n-hexyl acrylate | 57.63 | 21.92 | 20.44 |
| methacrylate | diacrylate (Mn = 700) | | | | |
| 2-hydroxyethyl | n-hexyl acrylate | poly(ethylene glycol) | 61.61 | 21.85 | 16.54 |
| methacrylate | | diacrylate (Mn = 700) | | | |
| 2-hydroxyethyl | n-hexyl acrylate | poly(ethylene glycol) | 65.63 | 23.28 | 11.10 |
| methacrylate | | diacrylate (Mn = 700) | | | |
| 2-hydroxyethyl | n-hexyl acrylate | tetraethylene glycol | 65.95 | 23.39 | 10.66 |
| methacrylate | | dimethacrylate | | | |
| 2-hydroxyethyl | N,N-dimethylacrylamide | ethylene glycol | 73.40 | 16.62 | 9.98 |
| methacrylate | | dimethacrylate | | | |

TABLE 2
Oxygen sensor compositions
(w/w % of monomer and/or polymer content of major components)

| | | | wt % | wt % | wt % |
|---|---|---|---|---|---|
| Component 1 | Component 2 | Component 3 | cmpt 1 | cmpt 2 | cmpt 3 |
| 2-hydroxyethyl | 2,2,3,3,4,4,5,5- | | 95.23 | 4.77 | |
| methacrylate | octafluoro- | | | | |
| | 1,6-hexyldimethacrylate | | | | |
| o-nitrobenzyl | tetraethylene glycol | | 90.98 | 9.02 | |
| methacrylate | dimethacrylate | | | | |
| poly(ethylene glycol) | Polyurethane D640 (5%) | | 78.89 | 21.11 | |
| diacrylate (Mn = 700) | | | | | |
| poly(ethylene glycol) | Polyurethane D640 (5%) | | 91.79 | 8.21 | |
| diacrylate (Mn = 700) | | | | | |
| poly(ethylene glycol) | Polyurethane D640 (5%) | | 81.65 | 18.35 | |
| diacrylamide (Mn = 3700) | | | | | |
| poly(ethylene glycol) | Polyurethane D640 (5%) | | 73.66 | 26.34 | |
| diacrylate (Mn = 700) | | | | | |
| o-nitrobenzyl | ethylene glycol | | 91.29 | 8.71 | |
| methacrylate | dimethacrylate | | | | |
| poly(ethylene glycol) | Polyurethane D640 (5%) | | 78.87 | 21.13 | |
| diacrylate (Mn = 700) | | | | | |
| poly(ethylene glycol) | Polyurethane D640 (5%) | | 83.29 | 16.71 | |
| diacrylate (Mn = 700) | | | | | |
| 2-hydroxyethyl | tetraethylene glycol | | 89.83 | 10.17 | |
| methacrylate | dimethacrylate | | | | |
| poly(ethylene glycol) | Polyurethane D640 | | 71.36 | 28.64 | |
| diacrylate (Mn = 700) | (10%) | | | | |
| 2-hydroxyethyl | tetraethylene glycol | | 95.15 | 4.85 | |
| methacrylate | dimethacrylate | | | | |
| 2-hydroxyethyl | tetraethylene glycol | | 69.61 | 30.39 | |
| methacrylate | dimethacrylate | | | | |
| 2-hydroxyethyl | tetraethylene glycol | | 94.91 | 5.09 | |
| methacrylate | dimethacrylate | | | | |
| 2-hydroxyethyl | tetraethylene glycol | | 97.96 | 2.04 | |
| methacrylate | dimethacrylate | | | | |
| 2,2,3,3,4,4,4- | ethylene glycol | | 96.31 | 3.69 | |
| heptafluorobutyl | dimethacrylate | | | | |
| methacrylate | | | | | |
| 2-hydroxyethyl | tetraethylene glycol | | 98.00 | 2.00 | |
| methacrylate | dimethacrylate | | | | |
| 2-(tert-butylamino)ethyl | ethylene glycol | | 94.40 | 5.60 | |
| methacrylate | dimethacrylate | | | | |
| 2-hydroxyethyl | ethylene glycol | | 95.10 | 4.90 | |
| methacrylate | dimethacrylate | | | | |
| 2,2,3,3,4,4,4- | ethylene glycol | | 83.93 | 16.07 | |
| heptafluorobutyl | dimethacrylate | | | | |
| methacrylate | | | | | |
| 2,2,3,4,4,4- | ethylene glycol | | 96.13 | 3.87 | |
| hexafluorobutyl | dimethacrylate | | | | |
| methacrylate | | | | | |
| acrylamide | N,N′- | | 99.86 | 0.14 | |
| | methylenebis(acrylamide) | | | | |
| 2,2,3,3,4,4,4- | poly(ethylene glycol) | | 82.77 | 17.23 | |
| heptafluorobutyl | diacrylate (Mn = 700) | | | | |
| methacrylate | | | | | |
| 2-hydroxyethyl | ethylene glycol | | 90.18 | 9.82 | |
| methacrylate | dimethacrylate | | | | |
| 1,1,1,3,3,3- | tetraethylene glycol | | 91.79 | 8.21 | |
| hexafluoroisopropyl | dimethacrylate | | | | |
| acrylate | | | | | |
| 2,2,3,3,4,4,4- | poly(ethylene glycol) | | 91.53 | 8.47 | |
| heptafluorobutyl | diacrylate (Mn = 700) | | | | |
| methacrylate | | | | | |
| 2,2,3,3,4,4,4- | ethylene glycol | | 96.13 | 3.87 | |
| heptafluorobutyl | dimethacrylate | | | | |
| methacrylate | | | | | |
| 2,2,2-trifluoroethyl | poly(ethylene glycol) | | 98.10 | 1.90 | |
| methacrylate | diacrylate (Mn = 700) | | | | |
| 2,2,3,3,4,4,4- | tetraethylene glycol | | 95.98 | 4.02 | |
| heptafluorobutyl | dimethacrylate | | | | |
| methacrylate | | | | | |
| 2,2,3,3-tetrafluoropropyl | poly(ethylene glycol) | | 95.45 | 4.55 | |
| methacrylate | diacrylate (Mn = 700) | | | | |
| 3-chloro-2-hydroxypropyl | tetraethylene glycol | | 98.20 | 1.80 | |
| methacrylate | dimethacrylate | | | | |
| 2,2,2- | poly(ethylene glycol) | | 95.25 | 4.75 | |
| trifluoroethyl methacrylate | diacrylate (Mn = 700) | | | | |
| 2,2,3,3,4,4,4- | trimethylolpropane | | 95.87 | 4.13 | |
| heptafluorobutyl | triacrylate | | | | |
| methacrylate | | | | | |
| 2,2,2-trifluoroethyl | ethylene glycol | | 98.25 | 1.75 | |
| methacrylate | dimethacrylate | | | | |
| 2,2,3,3-tetrafluoropropyl | ethylene glycol | | 91.53 | 8.47 | |
| methacrylate | dimethacrylate | | | | |
| 2,2,2-trifluoroethyl | ethylene glycol | | 86.66 | 13.34 | |
| methacrylate | dimethacrylate | | | | |
| 2,2,2-trifluoroethyl | tetraethylene glycol | | 98.18 | 1.82 | |
| methacrylate | dimethacrylate | | | | |
| 2,2,2-trifluoroethyl | ethylene glycol | | 91.17 | 8.83 | |
| methacrylate | dimethacrylate | | | | |
| 2-fluoroethyl methacrylate | poly(ethylene glycol) | | 69.43 | 30.57 | |
| | diacrylate (Mn = 700) | | | | |
| methyl methacrylate | ethylene glycol | | 94.47 | 5.53 | |
| | dimethacrylate | | | | |
| 2-fluoroethyl methacrylate | tetraethylene glycol | | 95.08 | 4.92 | |
| | dimethacrylate | | | | |
| 2,2,3,4,4,4- | ethylene glycol | | 88.12 | 11.88 | |
| hexafluorobutyl | dimethacrylate | | | | |
| methacrylate | | | | | |
| 2,2,3,4,4,4- | ethylene glycol | | 92.17 | 7.83 | |
| hexafluorobutyl | dimethacrylate | | | | |
| methacrylate | | | | | |
| 2,2,2-trifluoroethyl | tetraethylene glycol | | 95.44 | 4.56 | |
| methacrylate | dimethacrylate | | | | |
| 2,2,3,3,4,4,4- | 1,6-hexanediol diacrylate | | 96.94 | 3.06 | |
| heptafluorobutyl | | | | | |
| methacrylate | | | | | |
| methyl methacrylate | tetraethylene glycol | | 94.26 | 5.74 | |
| | dimethacrylate | | | | |
| methyl methacrylate | tetraethylene glycol | | 88.62 | 11.38 | |
| | dimethacrylate | | | | |
| 2-fluoroethyl methacrylate | tetraethylene glycol | | 98.03 | 1.97 | |
| | dimethacrylate | | | | |
| 2,2,2-trifluoroethyl | ethylene glycol | | 95.61 | 4.39 | |
| methacrylate | dimethacrylate | | | | |
| 2-fluoroethyl methacrylate | poly(ethylene glycol) | | 94.87 | 5.13 | |
| | diacrylate (Mn = 700) | | | | |
| 2,2,3,3,4,4,4- | poly(ethylene glycol) | | 95.80 | 4.20 | |
| heptafluorobutyl | diacrylate (Mn = 700) | | | | |
| methacrylate | | | | | |
| 2-fluoroethyl methacrylate | poly(ethylene glycol) | | 79.57 | 20.43 | |
| | diacrylate (Mn = 700) | | | | |
| 2,2,2-trifluoroethyl | 1,6-hexanediol diacrylate | | 96.69 | 3.31 | |
| methacrylate | | | | | |
| ethylene glycol | ethylene glycol | | 95.15 | 4.85 | |
| dicyclopentenyl ether | dimethacrylate | | | | |
| methacrylate | | | | | |
| 2,2,3,4,4,4- | ethylene glycol | | 92.90 | 7.10 | |
| hexafluorobutyl | dimethacrylate | | | | |
| methacrylate | | | | | |
| 2-fluoroethyl methacrylate | poly(ethylene glycol) | | 97.95 | 2.05 | |
| | diacrylate (Mn = 700) | | | | |
| 2-fluoroethyl methacrylate | poly(ethylene glycol) | | 82.96 | 17.04 | |
| | diacrylate (Mn = 700) | | | | |
| 2-fluoroethyl methacrylate | poly(ethylene glycol) | | 89.76 | 10.24 | |
| | diacrylate (Mn = 700) | | | | |
| 2,2,2-trifluoroethyl | trimethylolpropane | | 95.33 | 4.67 | |
| methacrylate | triacrylate | | | | |
| 2,2,2-trifluoroethyl | 1,6-hexanediol diacrylate | | 96.52 | 3.48 | |
| methacrylate | | | | | |
| ethylene glycol | tetraethylene glycol | | 4.97 | 5.03 | |
| dicyclopentenyl ether | dimethacrylate | | | | |
| methacrylate | | | | | |
| benzyl methacrylate | ethylene glycol | | 95.05 | 4.95 | |
| | dimethacrylate | | | | |
| 2-fluoroethyl methacrylate | ethylene glycol | | 95.26 | 4.74 | |
| | dimethacrylate | | | | |
| pentafluorobenzyl | ethylene glycol | | 92.27 | 7.73 | |
| methacrylate | dimethacrylate | | | | |
| pentafluorobenzyl | ethylene glycol | | 98.48 | 1.52 | |
| methacrylate | dimethacrylate | | | | |
| pentafluorobenzyl | ethylene glycol | | 96.18 | 3.82 | |
| methacrylate | dimethacrylate | | | | |
| pentafluorobenzyl | poly(ethylene glycol) | | 91.65 | 8.35 | |
| methacrylate | diacrylate (Mn = 700) | | | | |
| 2,2,3,3,4,4,4- | 2-hydroxyethyl | ethylene glycol | 53.69 | 41.98 | 4.33 |
| heptafluorobutyl | methacrylate | dimethacrylate | | | |
| methacrylate | | | | | |
| 2-carboxyethyl acrylate | [2-(acryloyloxy)ethyl]trimethylammonium | tetraethylene glycol | 59.00 | 38.88 | 2.12 |
| | chloride | dimethacrylate | | | |
| 2-carboxyethyl acrylate | [2-(acryloyloxy)ethyl]trimethylammonium | tetraethylene glycol | 57.08 | 37.62 | 5.30 |
| | chloride | dimethacrylate | | | |
| [2- | Polyurethane D640 | N,N′- | 83.64 | 16.24 | 0.12 |
| (methacryloyloxy)ethyl]dimethyl- | (10%) | methylenebis(acrylamide) | | | |
| (3-sulfopropyl)ammonium | | | | | |
| hydroxide | | | | | |
| 2-carboxyethyl acrylate | [2-(acryloyloxy)ethyl]trimethylammonium | tetraethylene glycol | 43.14 | 41.08 | 15.78 |
| | chloride | dimethacrylate | | | |
| 2-carboxyethyl acrylate | [2-(acryloyloxy)ethyl]trimethylammonium | tetraethylene glycol | 53.91 | 35.52 | 10.57 |
| | chloride | dimethacrylate | | | |
| acrylamide | Polyurethane D640 (5%) | N,N′- | 77.84 | 22.06 | 0.10 |
| | | methylenebis(acrylamide) | | | |
| [2- | Polyurethane D640 | N,N′- | 80.15 | 19.62 | 0.22 |
| (methacryloyloxy)ethyl]dimethyl- | (10%) | methylenebis(acrylamide) | | | |
| (3-sulfopropyl)ammonium | | | | | |
| hydroxide | | | | | |
| [2- | Polyurethane D640 (5%) | N,N′- | 77.84 | 22.06 | 0.10 |
| (methacryloyloxy)ethyl]dimethyl- | | methylenebis(acrylamide) | | | |
| (3-sulfopropyl)ammonium | | | | | |
| hydroxide/acrylamide | | | | | |
| (1:1) | | | | | |
| 2-carboxyethyl acrylate | [2-(acryloyloxy)ethyl]trimethylammonium | tetraethylene glycol | 47.62 | 31.38 | 21.01 |
| | chloride | dimethacrylate | | | |
| 2-hydroxyethyl | tetraethylene glycol | 2-methacryloyloxyethyl | 80.15 | 18.14 | 1.71 |
| methacrylate | dimethacrylate | phosphorylcholine | | | |
| 2-hydroxyethyl | tetraethylene glycol | 2-methacryloyloxyethyl | 96.63 | 2.68 | 0.69 |
| methacrylate | dimethacrylate | phosphorylcholine | | | |
| lauryl methacrylate | tetraethylene glycol | 2-methacryloyloxyethyl | 55.90 | 38.32 | 5.78 |
| | dimethacrylate | phosphorylcholine | | | |
| lauryl methacrylate | tetraethylene glycol | 2-methacryloyloxyethyl | 86.52 | 11.24 | 2.24 |
| | dimethacrylate | phosphorylcholine | | | |
| 2-hydroxyethyl | ethylene glycol | n-hexyl acrylate | 77.88 | 14.96 | 7.16 |
| methacrylate | dimethacrylate | | | | |
| 2-hydroxyethyl | 2-fluoroethyl | poly(ethylene glycol) | 75.23 | 19.50 | 5.27 |
| methacrylate | methacrylate | diacrylate (Mn = 700) | | | |
| 2-hydroxyethyl | N,N-dimethylacrylamide | ethylene glycol | 73.40 | 16.62 | 9.98 |
| methacrylate | | dimethacrylate | | | |
| 2,2,3,3,4,4,4- | ethylene glycol | 2,2,2-trifluoroethyl | 68.55 | 16.40 | 15.05 |
| heptafluorobutyl | dimethacrylate | methacrylate | | | |
| methacrylate | | | | | |
| 2-hydroxyethyl | ethylene glycol | hydroxypropyl | 76.85 | 14.76 | 8.40 |
| methacrylate | dimethacrylate | methacrylate | | | |
| 2-hydroxyethyl | n-hexyl acrylate | ethylene glycol | 78.47 | 12.99 | 8.54 |
| methacrylate | | dimethacrylate | | | |
| 2-hydroxyethyl | ethylene glycol | hydroxypropyl | 81.29 | 9.83 | 8.88 |
| methacrylate | dimethacrylate | methacrylate | | | |
| 2-hydroxyethyl | n-hexyl acrylate | ethylene glycol | 74.46 | 15.41 | 10.13 |
| methacrylate | | dimethacrylate | | | |
| 2-hydroxyethyl | hydroxypropyl | ethylene glycol | 63.42 | 26.73 | 9.86 |
| methacrylate | methacrylate | dimethacrylate | | | |
| 2-hydroxyethyl | ethylene glycol | 2-methacryloyloxyethyl | 80.71 | 17.57 | 1.72 |
| methacrylate | dimethacrylate | phosphorylcholine | | | |
| 2-hydroxyethyl | 2-fluoroethyl | poly(ethylene glycol) | 56.03 | 38.73 | 5.23 |
| methacrylate | methacrylate | diacrylate (Mn = 700) | | | |
| 2,2,2-trifluoroethyl | 2-hydroxyethyl | ethylene glycol | 78.13 | 17.39 | 4.48 |
| methacrylate | methacrylate | dimethacrylate | | | |
| 2-hydroxyethyl | methyl methacrylate | tetraethylene glycol | 47.45 | 41.81 | 10.74 |
| methacrylate | | dimethacrylate | | | |
| 2-fluoroethyl methacrylate | 2-hydroxyethyl | poly(ethylene glycol) | 48.25 | 46.53 | 5.22 |
| | methacrylate | diacrylate (Mn = 700) | | | |
| 2,2,3,4,4,4- | 2-hydroxyethyl | ethylene glycol | 80.30 | 15.66 | 4.04 |
| hexafluorobutyl | methacrylate | dimethacrylate | | | |
| methacrylate | | | | | |
| 2-fluoroethyl methacrylate | 2-hydroxyethyl | poly(ethylene glycol) | 76.41 | 18.42 | 5.16 |
| | methacrylate | diacrylate (Mn = 700) | | | |
| 2,2,2-trifluoroethyl | 2,2,3,3,4,4,4- | ethylene glycol | 74.51 | 21.21 | 4.28 |
| methacrylate | heptafluorobutyl | dimethacrylate | | | |
| | methacrylate | | | | |

TABLE 3
Combinations of tubing and coatings

| Tubing Size | | | | |
|---|---|---|---|---|
| (ID/OD, | | Number | Coating | |
| inch) | Tubing | of Coats | Concentration | Coating |
| | | 3 | 4.0% w/v in | PU SG-80A |
| | | | (EtOH/THF 1:1, v/v) | |
| 0.016/0.04 | TPX | | | |
| 0.007/0.014 | PU | | | |
| 0.012/0.025 | Silicone | | | |
| | | 3 | 5.0% w/v in | PU SG-80A |
| | | | (EtOH/THF 1:1, v/v) | |
| | | 5 | 5.0% w/v in | PU SG-80A |
| | | | (EtOH/THF 1:1, v/v) | |
| 0.016/0.04 | TPX | 1 | 5.0% w/v in | PU D3 |
| | | | (EtOH/H₂O, 9:1, v/v) | |
| | | 1 | 5.0% w/v in | PU SG-80A |
| | | | (EtOH/THF 1:1, v/v) | |
| | | 1 | 5.0% w/w in THF | PMMA |
| | | 1 | 5.0% w/w in THF | PCL |
| | | 3 | 5.0% w/w in THF | PMMA |
| | | 3 | 5.0% w/w in THF | PU OP770 |
| 0.016/0.04 | PU | | | |
| 0.015/0.043 | PE | | | |
| 0.034/0.05 | PE | | | |
| | | 3 | 5% w/w in | Polycarbonate |
| | | | Methylene chloride | |
| | | | (CH2 Cl2) | |
| | | 5 | 5% w/w in CH2 Cl2 | Polycarbonate |
| | | 1 | 5.0% w/w in THF | PU OP770 |
| | | 2 | 5.0% w/w in THF | PU OP770 |
| 0.034/0.05 | PE | 2 | 5.0% w/w in THF | PU OP770 |
| | | 1 | 2.5% w/w in THF | PU OP770 |
| | | 1 | 7.5% w/w in THF | PU OP770 |
| | | 1 | 10% w/w in THF | PU OP770 |
| | | 1 | 5.0% w/w in CH2 | Polycarbonate |
| | | | Cl2 | |
| | | 1 | 10.0% w/w in CH2 | Polycarbonate |
| | | | Cl2 | |
| | | 1 | 15.0% w/w in CH2 | Polycarbonate |
| | | | Cl2 | |
| | | 2 | 5.0% w/v in | PU SG-80A |
| | | | (EtOH/THF 1:1, v/v) | |
| | | 1 | 5% w/w in CH2 Cl2 | PU SG-80A |
| | | 1 | 5.0% w/w in THF | PU SG-85A |
| | | 1 | 5.0% w/w in THF | PU EG-93A |
| | | 5 | 5.0% w/w in THF | PU SG-85A |
| | | 3 | 5.0% w/w in THF | PU SG-85A |
| | | 2 | 5.0% w/w in THF | PU SG-85A |
| 0.028/0.04 | TPX | 1 | 5.0% w/v in | PU SG-80A |
| | | | (EtOH/THF 1:1, v/v) | |
| 0.016/0.04 | TPX | 1 | 5.0% w/v in | PU SG-80A |
| | | | (EtOH/THF 1:1, v/v) | |
| 0.016/0.04 | TPX | 3 | 5.0% w/v in | PU SG-80A |
| | | | (EtOH/THF 1:1, v/v) | |
| 0.016/0.04 | TPX | 5 | 5.0% w/v in | PU SG-80A |
| | | | (EtOH/THF 1:1, v/v) | |
| 0.016/0.04 | TPX | 3 | 5.0% w/w in THF | PU SG-85A |
| 0.023/0.038 | PE | 3 | 5.0% w/w in THF | PU EG-93A |
| 0.034/0.05 | PE | 3 | 5.0% w/w in THF | PU EG-93A |
| 0.023/0.038 | PU | 3 | 5.0% w/w in THF | PU EG-93A |
| 0.016/0.04 | TPX | 3 | 5.0% w/w in THF | PU EG-93A |

### Example 1

### Preparation of First Sensing Layer Including Lactate Oxidase of a Layered Lactate Sensor

TABLE 4
Components for layered sensors in Example I-III

| | Total | Component 1/ | | | |
|---|---|---|---|---|---|
| | Volume | Component 2/ | | | Enzymatic |
| Formulation | (uL) | Component 3 | Cosolvents | Dye | Components (w/v %) |
| Lactate Sensor | 500 | HEMA/HPMA/EGDMA | 0.67M 1-Methyl-2- | 1 mM Pd-BP- | 2.1% (w/v) LOx from |
| | | (63.4/26.7/9.8% w/w of | pyrrolidinone (NMP) | AEME-4 in NMP | Aerococcus viridans |
| | | monomer and/or polymer | | | |
| | | content of major | | | |
| | | components) | | | |
| Coating | 200 | 0.88 mM polycarbonate in | 15.7M Methylene | NA | NA |
| | | methylene chloride | chloride | | |
| Oxygen Sensor | 230 | PUD640 (5% wt/v in 9:1 | 0.45M NMP | 1.3 mM Pd-BP- | NA |
| | | ethanol/water)/PEGDA700 | | AEME-4 in NMP | |
| | | (16.7/83.3% w/w of | | | |
| | | polymer content only) | | | |

The first sensing layer, including lactate oxidase, of a layered lactate sensor was prepared as follows (Table 4): Irgacure 651 (Sigma-Aldrich, HEMA (Polysciences), HPMA (Sigma-Aldrich), EGDMA (Sigma-Aldrich), Pd-BMAP-AEME-4 (U.S. Pat. No. 9,375,494, which is hereby incorporated by reference herein in its entirety), and NMP (N-Methyl-2-pyrrolidone, Sigma-Aldrich) were added together and mixed well to form solution 1. 2-Aminoethylmethacrylate hydrochloride (AEMA, Sigma-Aldrich), LOx (Lactate Oxidase, Sekisui) from Aerococcus viridans, and PBS (phosphate buffered saline, 20 mM) were mixed together to form solution 2. Solution 1 was added to solution 2 to get a mixture with final concentrations of Irgacure 651 (19.5 mM), HEMA (3.63 M), HPMA (1.35 M), EGDMA (0.37 M), AEMA (0.56 mM in water), Pd-BMAP-AEME-4 (1 mM), NMP (0.67 M) and enzymatic components (LOx, 2.1% wt/v) in 20 mM PBS such that the PBS volume was 18.8% of the total volume mixture. The mixture was polymerized and prepared for the coating process.

Pd-BP-AEME-4 has the following structure:

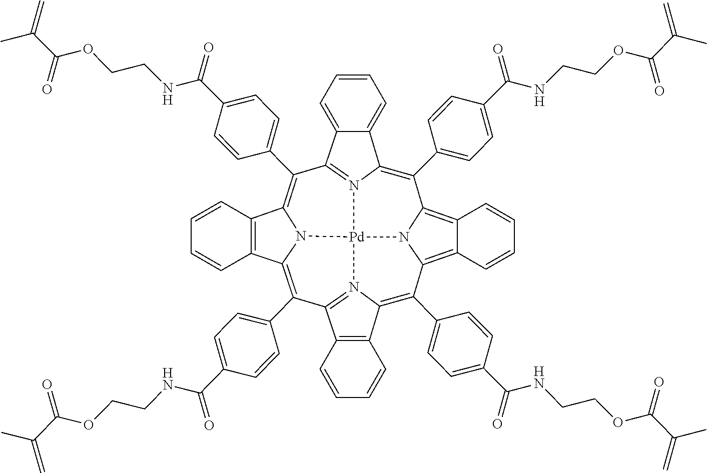

Additional first sensing layers including lactate oxidase were prepared as described above, using the monomers and/or polymers shown in Table 1.

### Example 2

### Application of a Coating to the First Sensing Layer Including Lactate Oxidase of a Layered Lactate Sensor

A coating was applied to the first sensing layer including lactate oxidase prepared above. Water on the surface of the lactate sensing layer was removed. The sensing layers were coated with a polycarbonate solution ((VWR) 0.88 mM in methylene chloride (Sigma-Aldrich)) and dried. After coating, the sensors were stored in PBS (20 mM) solution.

Additional passive layers were prepared as described above, using the tubing and coatings shown in Table 3.

### Example 3

### Application of a Second Sensing Layer, Functioning as a Reference, to the Coating on First Sensing Layer, Forming the Layered Lactate Sensor

A second sensing layer, functioning as a reference, was applied to the coating on the first sensing layer prepared above.

Irgacure 651 (19.5 mM), PEGDA700 (poly(ethylene glycol) diacrylate average Mn 700, 83.3% w/w of polymer content only, Sigma-Aldrich), Pd-BMAP-AEME-4 (1.3 mM, prepared as described above), NMP (0.45M), and PU D640 (5 wt/v % in ethanol/water 9:1 v/v, 16.7% w/w of polymer content only, AdvanSource Biomaterials Inc.) were mixed such that the ethanol/water solution was 72% (v/v) to form the oxygen reference layer (solution 3) solution. To incorporate the oxygen reference solution on the passive layer, the water on the surface of the passive layer was removed. The coating was then applied to the surface. Coated sensors were then stored in PBS.

Additional second sensing layers including oxygen sensors were prepared as described above in both Example I and III, using the monomers and/or polymers shown in Table 2.

The formulation of the layered lactate sensors made in Examples I-III is summarized in Table 4.

Additional formulations of layered lactate/oxygen and oxygen/oxygen sensors are shown in Tables 6 and 7. Displayed in the tables are the weight percentages of the major monomer and/or polymer components with respect to each other. FIGS. 4A-4C show additional sensing layer 1 configurations. Sensors A-K in Tables 6 and 7 and Examples I-III above represent sensor configuration shown in FIG. 4A. FIG. 4B shows sensing layer 1 in two separate regions, but both regions are fabricated polymers containing sensing recognition elements. Sensors L-Q in Tables 6 and 7 represent sensor configuration shown in FIG. 4B. FIG. 4C shows a configuration where sensing layer 1 may contain a polymer region surrounded by a non-polymer component and both regions contain sensing recognition elements. Sensor R in Tables 6 and 7 represent sensor configuration shown in FIG. 4C.

### Example 4

### Performance of the Layered Lactate Sensor

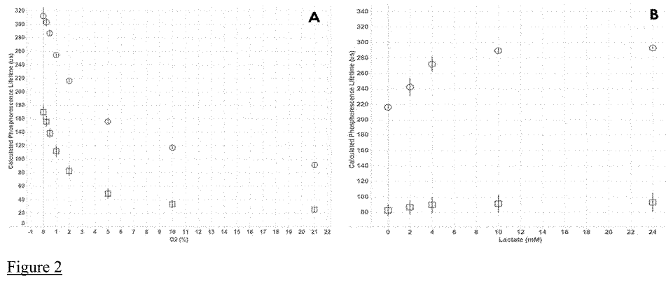

The performance of the layered lactate sensors prepared in Examples I-III above were tested and the data is shown in FIG. 2.

Layered lactate sensors were placed in a customized test fixture with controllable oxygen levels. All sensors were tested in 500 ml of PBS and allowed to equilibrate at 37° C. An oxygen and lactate modulation were performed sequentially on the sensors. Automated gas mixing systems and pumps were used to modulate oxygen concentration and dispense lactate at stepwise increases in concentration, respectively. Sensors were tested at 0, 0.25, 0.5, 1, 2, 5, 10, 21% oxygen and 0, 2, 4, 10, 24 mM lactate at a fixed 2% oxygen. At each oxygen and lactate concentration, the sensor phosphorescence signal was equilibrated and phosphorescent lifetimes from each sensing portion was calculated using custom algorithms. Response curves were generated by averaging the phosphorescence signal of the last 2 minutes of each step prior to changes in either oxygen or lactate.

### Example 5

TABLE 5
Components for layered sensors in Example V

| | Total | | | | |
|---|---|---|---|---|---|
| | Volume | Component 1/Component | | | Enzymatic |
| Formulation | (uL) | 2/Component 3 | Cosolvents | Dye | Components (w/v %) |
| Lactate | 500 | HEMA/HPMA/EGDMA | 0.67M 1- | NA | 2.1% (w/v) LOx from |
| Sensor | | (63.4/26.7/9.8% w/w of | Methyl-2- | | Aerococcus viridans |
| | | monomer and/or polymer | pyrrolidinone | | |
| | | content of major components) | (NMP) | | |
| Coating | 200 | EG-93A (5%, w/w) in THF | 12.3M | NA | NA |
| | | | Tetrahydrofuran | | |
| Oxygen | 250 | PUD640 (5% wt/v in 9:1 | 1.25M NMP | 1.2 mM Pd-BP- | NA |
| Sensor | | ethanol/water)/PEGDA700 | | AEMA-4 in | |
| | | (16.7/83.3% w/w of polymer | | NMP | |
| | | content only) | | | |

The passive layer serves multiple purposes. In this example, the passive layer serves to minimize cross-talk between sensing layer 1 (lactate sensor) and sensing layer 2 (oxygen sensor). The consumption of oxygen by lactate oxidase in sensing layer 1 may artificially change the reading of sensing layer 2 (oxygen sensor). Similarly stated, the passive layer is configured to isolate the reaction occurring and/or reactants consumed/products produced in layer 1 from reaching and/or influencing layer 2. Details for the sensing and passive layers are in Table 5. Briefly, EG-93A was dissolved in tetrahydrofuran (THF) at a concentration of 5% (w/w) for the passive layer. For the oxygen sensing layer, Irgacure 651 (19.5 mM), PEGDA700 (83.3% w/w of polymer content only), Pd-BP-AEME-4 (1.2 mM), NMP (1.25M), and PU D640 (5 wt/v % in ethanol/water 9:1 v/v, 16.7% w/w of polymer content only) were mixed such that the ethanol/water solution was 72% (v/v) of the total oxygen sensing solution. The lactate sensing layer, including lactate oxidase, of a layered lactate sensor was prepared as follows. Irgacure 651, HEMA, HPMA, EGDMA (ethylene glycol-dimethacrylate), and NMP (N-Methyl-2-pyrrolidone) were added together and mixed well to form solution 1. AEMA, LOx from Aerococcus viridans, and PBS (phosphate buffered saline, 20 mM) were mixed together to form solution 2. Solution 1 was added to solution 2 to get a mixture with final concentrations of Irgacure 651 (19.5 mM), HEMA (3.63 M), HPMA (1.35 M), EGDMA (0.37 M), AEMA (0.56 mM in water), NMP (0.67 M) and enzymatic component (LOx, 2.1 wt/v %) in 20 mM PBS such that the PBS volume was 18.8% of the total volume mixture. The mixture was polymerized and prepared for the coating process. The lactate sensing layer was wiped to get rid of water on surface and then coated with EG-93A solution. Additional layers were added to obtain sensors with 0, 1, and 3 layers. The sensing and passive layers were coated with the oxygen solution. Coated sensors were then stored in PBS.

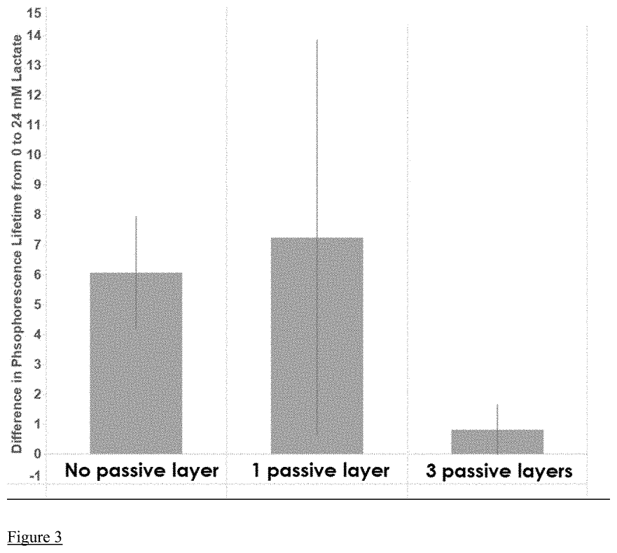

FIG. 3 shows the change in phosphorescent lifetime measurements from the oxygen sensing layer between 0 and 24 mM lactate. As the number of layers increases, the response of the oxygen sensor decreases close to zero indicating a small amount of cross-sensitivity impacting the oxygen sensing layer.

TABLE 6
Oxygen/oxygen sensor compositions (w/w % of monomer
and/or polymer content of major components)

| Passive Tubing | | Number of | | | |
|---|---|---|---|---|---|
| Layer (ID/OD, | | Passive | Sensing | | |
| inch) | Passive Layer | Layer Coats | Layer | Component 1 | Component 2 |
| | EG-93A | 1 | Oxygen | 2-hydroxyethyl | hydroxypropyl |
| | (5% w/w | | Sensing | methacrylate | methacrylate |
| | in THF) | | Layer 1 | | |
| | | 1 | Oxygen | Polyurethane | poly(ethylene |
| | | | Sensing | D640 (5%) | glycol) diacrylate |
| | | | Layer 2 | | |
| | EG-93A | 1 | Oxygen | 2-hydroxyethyl | hydroxypropyl |
| | (5% w/w | | Sensing | methacrylate | methacrylate |
| | in THF) | | Layer 1 | | |
| | | 1 | Oxygen | Polyurethane | poly(ethylene |
| | | | Sensing | D640 (5%) | glycol) diacrylate |
| | | | Layer 2 | | |
| | SG-80A | 2 | Oxygen | 2-hydroxyethyl | hydroxypropyl |
| | (5% w/v | | Sensing | methacrylate | methacrylate |
| | in 1:1 | | Layer 1 | | |
| | THF:EtOH) | 2 | Oxygen | Polyurethane | poly(ethylene |
| | | | Sensing | D640 (5%) | glycol) diacrylate |
| | | | Layer 2 | | |
| | SG-80A | 2 | Oxygen | 2-hydroxyethyl | hydroxypropyl |
| | (5% w/v | | Sensing | methacrylate | methacrylate |
| | in 1:1 | | Layer 1 | | |
| | THF:EtOH) | 2 | Oxygen | Polyurethane | poly(ethylene |
| | | | Sensing | D640 (5%) | glycol) diacrylate |
| | | | Layer 2 | | |
| | SG-80A | 1 | Oxygen | 2-hydroxyethyl | hydroxypropyl |
| | (5% w/v | | Sensing | methacrylate | methacrylate |
| | in 1:1 | | Layer 1 | | |
| | THF:EtOH) | 1 | Oxygen | Polyurethane | poly(ethylene |
| | | | Sensing | D640 (5%) | glycol) diacrylate |
| | | | Layer 2 | | |
| | EG-93A | 1 | Oxygen | 2-hydroxyethyl | hydroxypropyl |
| | (5% w/w | | Sensing | methacrylate | methacrylate |
| | in THF) | | Layer 1 | | |
| | | 1 | Oxygen | Polyurethane | poly(ethylene |
| | | | Sensing | D640 (5%) | glycol) diacrylate |
| | | | Layer 2 | | |
| | EG-93A | 1 | Oxygen | 2-hydroxyethyl | hydroxypropyl |
| | (5% w/w | | Sensing | methacrylate | methacrylate |
| | in THF) | | Layer 1 | | |
| | | 1 | Oxygen | Polyurethane | poly(ethylene |
| | | | Sensing | D640 (5%) | glycol) diacrylate |
| | | | Layer 2 | | |
| | PC (5% | 3 | Oxygen | 2-hydroxyethyl | hydroxypropyl |
| | w/w in | | Sensing | methacrylate | methacrylate |
| | CH2Cl2) | | Layer 1 | | |
| | | 3 | Oxygen | Polyurethane | poly(ethylene |
| | | | Sensing | D640 (5%) | glycol) diacrylate |
| | | | Layer 2 | | |
| | PC (5% | 5 | Oxygen | 2-hydroxyethyl | hydroxypropyl |
| | w/w in | | Sensing | methacrylate | methacrylate |
| | CH2Cl2) | | Layer 1 | | |
| | | 5 | Oxygen | Polyurethane | poly(ethylene |
| | | | Sensing | D640 (5%) | glycol) diacrylate |
| | | | Layer 2 | | |
| 0.016/0.04 | SG-80A | 1 | Oxygen | 2-hydroxyethyl | hydroxypropyl |
| polymethylpentene | (5% w/v | | Sensing | methacrylate | methacrylate |
| | in 1:1 | | Layer 1 | | |
| | THF:EtOH) | 1 | Oxygen | Polyurethane | poly(ethylene |
| | | | Sensing | D640 (5%) | glycol) diacrylate |
| | | | Layer | | |
| 0.016/0.04 | SG-80A | 3 | Oxygen | 2-hydroxyethyl | hydroxypropyl |
| polymethylpentene | (5% w/v | | Sensing | methacrylate | methacrylate |
| | in 1:1 | | Layer 1 | | |
| | THF:EtOH) | 3 | Oxygen | Polyurethane | poly(ethylene |
| | | | Sensing | D640 (5%) | glycol) diacrylate |
| | | | Layer 2 | | |
| | EG-93A | 2 | Oxygen | 2-hydroxyethyl | hydroxypropyl |
| | (5% w/w | | Sensing | methacrylate | methacrylate |
| | in THF) | | Layer 1 | | |
| | | 2 | Oxygen | Polyurethane | poly(ethylene |
| | | | Sensing | D640 (5%) | glycol) diacrylate |
| | | | Layer 2 | | |
| 0.016/0.04 | EG-93A | 3 | Oxygen | 2-hydroxyethyl | N,N- |
| polymethylpentene | (5% w/w | | Sensing | methacrylate | dimethylacrylamide |
| | in THF) | | Layer 1 | | |
| | | 3 | Oxygen | Polyurethane | poly(ethylene |
| | | | Sensing | D640 (5%) | glycol) diacrylate |
| | | | Layer 2 | | |
| 0.016/0.04 | EG-93A | 3 | Oxygen | 2-hydroxyethyl | ethylene glycol |
| polymethylpentene | (5% w/w | | Sensing | methacrylate | dimethacrylate |
| | in THF) | | Layer 1 | | |
| | | 3 | Oxygen | Polyurethane | poly(ethylene |
| | | | Sensing | D640 (5%) | glycol) diacrylate |
| | | | Layer 2 | | |
| 0.023/0.038 | EG-93A | 3 | Oxygen | 2-hydroxyethyl | N,N- |
| polyethylene | (5% w/w | | Sensing | methacrylate | dimethylacrylamide |
| | in THF) | | Layer 1 | | |
| | | 3 | Oxygen | Polyurethane | poly(ethylene |
| | | | Sensing | D640 (5%) | glycol) diacrylate |
| | | | Layer 2 | | |
| 0.016/0.04 | EG-93A | 3 | Oxygen | 2-hydroxyethyl | N,N- |
| polymethylpentene | (5% w/w | | Sensing | methacrylate | dimethylacrylamide |
| | in THF) | | Layer 1 | | |
| | | 3 | Oxygen | Polyurethane | poly(ethylene |
| | | | Sensing | D640 (5%) | glycol) diacrylate |
| | | | Layer 2 | | |
| 0.023/0.038 | EG-93A | 3 | Oxygen | 2-hydroxyethyl | hydroxypropyl |
| polyethylene | (5% w/w | | Sensing | methacrylate | methacrylate |
| | in THF) | | Layer 1 | | |
| | | 3 | Oxygen | Polyurethane | poly(ethylene |
| | | | Sensing | D640 (5%) | glycol) diacrylate |
| | | | Layer 2 | | |
| 0.016/0.04 | SG-80A | 1 | Oxygen | PBS | |
| polymethylpentene | (5% w/v | | Sensing | | |
| | in 1:1 | | Layer 1 | | |
| | THF:EtOH) | 1 | Oxygen | Polyurethane | poly(ethylene |
| | | | Sensing | D640 (5%) | glycol) diacrylate |
| | | | Layer 2 | | |

| | | | | | | | Ratio of |
|---|---|---|---|---|---|---|---|
| Passive Tubing | | | | | | | tau0 for |
| Layer (ID/OD, | | wt % | wt % | wt % | Dye or | tau0 | Layer 1/ |
| inch) | Component 3 | cmpt 1 | cmpt 2 | cmpt 3 | Sensor | (us) | Layer 2 |
| | ethylene | 63.42 | 26.73 | 9.86 | Pd-BP- | 304.08 | 2.32 |
| | glycol | | | | AEME-4 | | |
| | dimethacrylate | | | | | | |
| | | 16.71 | 83.29 | | Pd-BP- | 130.93 | |
| | | | | | AEME-1 | | |
| | ethylene | 63.42 | 26.73 | 9.86 | Pd-BP- | 311.98 | 1.66 |
| | glycol | | | | AEME-4 | | |
| | dimethacrylate | | | | | | |
| | | 16.71 | 83.29 | | Pd-BP- | 187.76 | |
| | | | | | AEME-4 | | |
| | ethylene | 63.42 | 26.73 | 9.86 | Pd-BP- | 313.13 | 1.83 |
| | glycol | | | | AEME-4 | | |
| | dimethacrylate | | | | | | |
| | | 16.71 | 83.29 | | Pd-BP- | 171.33 | |
| | | | | | AEME-4 | | |
| | ethylene | 63.42 | 26.73 | 9.86 | Pd-BP- | 315.64 | 1.81 |
| | glycol | | | | AEME-4 | | |
| | dimethacrylate | | | | | | |
| | | 16.71 | 83.29 | | Pd-BP- | 174.61 | |
| | | | | | AEME-4 | | |
| | ethylene | 63.42 | 26.73 | 9.86 | Pd-BP- | 282.33 | 1.89 |
| | glycol | | | | AEME-4 | | |
| | dimethacrylate | | | | | | |
| | | 16.71 | 83.29 | | Pd-BP- | 149.26 | |
| | | | | | AEME-4 | | |
| | ethylene | 63.42 | 26.73 | 9.86 | Pd-BP- | 316.04 | 1.96 |
| | glycol | | | | AEME-4 | | |
| | dimethacrylate | | | | | | |
| | | 16.71 | 83.29 | | Pd-BP- | 161.02 | |
| | | | | | AEME-4 | | |
| | ethylene | 63.42 | 26.73 | 9.86 | Pd-BP- | 295.42 | 2.27 |
| | glycol | | | | AEME-4 | | |
| | dimethacrylate | | | | | | |
| | | 16.71 | 83.29 | | Pd-BP- | 130.13 | |
| | | | | | AEME-4 | | |
| | ethylene | 63.42 | 26.73 | 9.86 | Pd-BP- | 328.34 | 1.74 |
| | glycol | | | | AEME-4 | | |
| | dimethacrylate | | | | | | |
| | | 16.71 | 83.29 | | Pd-BP- | 188.27 | |
| | | | | | AEME-4 | | |
| | ethylene | 63.42 | 26.73 | 9.86 | Pd-BP- | 330.33 | 1.75 |
| | glycol | | | | AEME-4 | | |
| | dimethacrylate | | | | | | |
| | | 16.71 | 83.29 | | Pd-BP- | 188.33 | |
| | | | | | AEME-4 | | |
| 0.016/0.04 | ethylene | 63.42 | 26.73 | 9.86 | Pd-BP- | 334.30 | 1.64 |
| polymethylpentene | glycol | | | | AEME-4 | | |
| | dimethacrylate | | | | | | |
| | | 21.13 | 78.87 | | Pd-BP- | 204.31 | |
| | | | | | AEME-4 | | |
| 0.016/0.04 | ethylene | 63.42 | 26.73 | 9.86 | Pd-BP- | 308.35 | 1.69 |
| polymethylpentene | glycol | | | | AEME-4 | | |
| | dimethacrylate | | | | | | |
| | | 21.13 | 78.87 | | Pd-BP- | 182.42 | |
| | | | | | AEME-4 | | |
| | ethylene | 63.42 | 26.73 | 9.86 | Oxygen | 352.63 | 2.13 |
| | glycol | | | | Sensor | | |
| | dimethacrylate | | | | | | |
| | | 16.71 | 83.29 | | Pd-BP- | 165.75 | |
| | | | | | AEME-1 | | |
| 0.016/0.04 | ethylene | 73.40 | 16.62 | 9.98 | Oxygen | 361.11 | 1.92 |
| polymethylpentene | glycol | | | | Sensor | | |
| | dimethacrylate | | | | | | |
| | | 21.13 | 78.87 | | Pd-BP- | 187.84 | |
| | | | | | AEME-4 | | |
| 0.016/0.04 | | 90.18 | 9.82 | | Oxygen | 371.29 | 1.94 |
| polymethylpentene | | | | | Sensor | | |
| | | 21.13 | 78.87 | | Pd-BP- | 191.76 | |
| | | | | | AEME-4 | | |
| 0.023/0.038 | ethylene | 73.40 | 16.62 | 9.98 | Oxygen | 364.32 | 1.96 |
| polyethylene | glycol | | | | Sensor | | |
| | dimethacrylate | | | | | | |
| | | 21.13 | 78.87 | | Pd-BP- | 186.23 | |
| | | | | | AEME-4 | | |
| 0.016/0.04 | ethylene | 73.40 | 16.62 | 9.98 | Oxygen | 328.61 | 1.85 |
| polymethylpentene | glycol | | | | Sensor | | |
| | dimethacrylate | | | | | | |
| | | 21.13 | 78.87 | | Pd-BP- | 177.23 | |
| | | | | | AEME-4 | | |
| 0.023/0.038 | ethylene | 63.42 | 26.73 | 9.86 | Oxygen | 340.30 | 1.90 |
| polyethylene | glycol | | | | Sensor | | |
| | dimethacrylate | | | | | | |
| | | 21.13 | 78.87 | | Pd-BP- | 178.93 | |
| | | | | | AEME-4 | | |
| 0.016/0.04 | | | | | Oxygen | 382.74 | 1.92 |
| polymethylpentene | | | | | Sensor | | |
| | | 21.13 | 78.87 | | Pd-BP- | 199.17 | |
| | | | | | AEME-4 | | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. For example, although some embodiments discussed above describe layers of sensors encapsulating underlying layers, it should be understood that other configurations are possible. For example, a passive layer of a sensor can be disposed between a first active layer and a second active layer longitudinally such that the passive layer separates the first active layer and the second active layer without any layer encapsulating any other. It should be understood that various alternatives to the embodiments of the disclosure described herein may be employed in practicing the disclosure. It is intended that the following claims define the scope of the disclosure and that methods and structures within the scope of these claims and their equivalents be covered thereby. Where methods described above indicate certain events occurring in certain order, the ordering of certain events may be modified. Additionally, certain of the events may be performed concurrently in a parallel process when possible, as well as performed sequentially as described above.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. An apparatus, comprising:
a first layer configured to produce a first signal associated with a concentration of a first analyte;
a second layer configured to produce a second signal associated with a concentration of a second analyte; and
a passive layer separating the first layer and the second layer.

2. The apparatus of claim 1, wherein the passive layer encapsulates the first layer.

3. The apparatus of claim 1, wherein the second layer encapsulates the passive layer.

4. The apparatus of claim 1, wherein the first layer is configured to produce the first signal based on the concentration of the first analyte in tissue of user when the apparatus is implanted in the user.

5. The apparatus of claim 1, wherein the first analyte and the second analyte are the same analyte.

6. The apparatus of claim 1, wherein the first analyte and the second analyte are different analytes.

7. The apparatus of claim 1, wherein:
the first signal is a first optical signal; and
the second signal is a second optical signal optically distinguishable from the first optical signal.

8. The apparatus of claim 1, wherein:
the first layer includes optically detectable dye; and
the first layer includes a first polymer.

9. The apparatus of claim 1, wherein:
the first layer includes a sensing moiety;
the first layer is configured to produce the first signal based on a change in concentration of the second analyte in the first layer based on a reaction between the first analyte and the sensing moiety.

10. The apparatus of claim 1, wherein:
the first layer includes a sensing moiety;
the first layer is configured to produce the first signal based on a change in concentration of the second analyte in the first layer based on a reaction between the first analyte and the sensing moiety;
the passive layer is configured to isolate the reaction from the second layer; and
the second signal is a reference signal associated with a concentration of the second analyte

11. The apparatus of claim 1, wherein:
the first analyte is lactate;
the second analyte is oxygen;
the first layer includes lactate oxidase;
the first layer is configured to produce the first signal based on a change in a concentration of oxygen based on a reaction between lactate and lactate oxidase; and
the second signal is a reference signal produced by the second layer associated with a concentration of oxygen.

12. The apparatus of claim 1, wherein the passive layer is configured to prevent at least one of an altered concentration of reactants or reaction products associated with the detection of the first analyte from reaching the second layer.

13. The apparatus of claim 1, wherein:
the first layer is configured to produce the first signal when a concentration of the first analyte is above a first threshold concentration; and
the second layer is configured to produce the second signal when a concentration of the second analyte is above a second threshold concentration greater than the first threshold concentration; and
the first analyte and the second analyte are the same analyte.

14. The apparatus of claim 1, wherein:
the first layer includes a first sensing moiety sensitive to the first analyte at concentrations between a first threshold concentration and a second threshold concentration, the first sensing moiety being saturated when a concentration of the first analyte exceeds the second threshold concentration;
the second layer includes a second sensing moiety sensitive to the second analyte at concentrations between a third threshold concentration and a fourth threshold concentration, the third threshold concentration being greater than the first threshold concentration, the fourth threshold concentration being greater than the second threshold concentration; and
the first analyte and the second analyte are the same analyte.

15. An apparatus, comprising:
a sensor configured to be disposed in a body of a user, the sensor including:
a first layer configured to produce a reference signal based on a concentration of a first analyte in tissue of the user;
a second layer configured to produce a measurement signal associated with a concentration of a second analyte in tissue of the user, the measurement signal dependent on the concentration of the first analyte; and
a passive layer isolating the first layer from the second layer.

16. The apparatus of claim 15, wherein the passive layer is configured to prevent a local concentration of the first analyte in the second layer from being detected by the first layer.

17. The apparatus of claim 15, wherein the second layer includes lactate oxidase and a sensing moiety configured to produce the measurement signal associated with a concentration of lactate.

18. The apparatus of claim 15, wherein:
the first layer and the second layer include a common luminescent dye configured to produce the reference signal and the measurement signal;
the first layer includes a first polymer bound to the luminescent dye that is configured to alter a decay rate of the luminescent dye such that the reference signal has a first characteristic duration; and
the second layer includes a second polymer bound to the luminescent dye that is different from the first polymer, the second polymer configured to alter the decay rate of the luminescent dye such that the measurement signal has a second characteristic duration different from the first characteristic duration.

19. The apparatus of claim 15, wherein:
the first layer and the second layer each include a sensing moiety configured to emit an optical signal having a common characteristic wavelength;
the first layer includes a first polymer bound to the sensing moiety that is configured to alter the common characteristic wavelength such that the reference signal has a first characteristic wavelength; and
the second layer includes a second polymer bound to the sensing moiety that is different from the first polymer, the second polymer configured to alter the common characteristic wavelength such that the measurement signal has a second characteristic wavelength different from the first characteristic wavelength.

20. A method, comprising:
polymerizing a first precursor solution to form a first layer of a sensor, the first precursor solution including a first sensing moiety configured to emit a first optical signal associated with a concentration of a first analyte;
encapsulating the first layer of the sensor with a passive layer; and
polymerizing a second precursor solution to form a second layer of the sensor, the second precursor solution including a second sensing moiety configured to emit a second optical signal associated with a concentration of a second analyte.

21. The method of claim 20, wherein:
the first layer is encapsulated with the passive layer after the first precursor solution is polymerized; and
the second layer is polymerized to encapsulate the passive layer after the first layer is encapsulated with the passive layer.

22. The method of claim 20, wherein the first layer is polymerized inside the passive layer, which is pre-formed before the first layer is polymerized.

23. The method of claim 20, wherein the first layer is polymerized inside the passive layer, which is pre-formed before the first layer is polymerized such that the passive layer partially encapsulates the first layer, the method further comprising:
sealing the passive layer such that the passive layer completely encapsulates the first layer.
